(19) United States
(12) Patent Application Publication
(10) Pub. No.: US 2018/0067097 A1
(43) Pub. Date: Mar. 8, 2018

(54) MOLECULAR MARKERS AND METHODS FOR SAMPLE ANALYSIS VIA MASS SPECTROMETRY

(71) Applicant: Board of Regents, The University of Texas System, Austin, TX (US)

(72) Inventors: Livia S. EBERLIN, Austin, TX (US); Jialing ZHANG, Austin, TX (US)

(73) Assignee: Board of Regents, The University of Texas System, Austin, TX (US)

(21) Appl. No.: 15/648,276

(22) Filed: Jul. 12, 2017

Related U.S. Application Data
(60) Provisional application No. 62/361,855, filed on Jul. 13, 2016.

(51) Int. Cl. G01N 33/483; G06F 19/00; H01J 49/26

(52) U.S. Cl. CPC G01N 33/4833; H01J 49/26; G06F 19/34

(57) ABSTRACT

Methods for detecting cancer cells, or aggressive cancers, by measuring levels of cardiolipin molecules are provided. Methods of treating identified cancers are likewise provided

This application claims the benefit of U.S. Provisional Patent Application No. 62/361,855, filed Jul. 13, 2016, the entirety of which is incorporated herein by reference.

This invention was made with government support under Grant no. R00 CA190783 awarded by the National Institutes of Health. The government has certain rights in the invention.

### BACKGROUND OF THE INVENTION

### 1. Field of the Invention

The present invention relates generally to the field of molecular biology, organic chemistry and oncology. More particularly, it concerns lipid markers for disease lesions, such as cancer.

### 2. Description of Related Art

Recent years have seen a wide array of biological markers identified that can be used for diagnosis and prognosis of cancers. However, the wide range of genetic markers identified to date are still unable, in many cases, to provide accurate information to identify potential cancers, particularly aggressive cancers. Oncocytic tumors, for example, are a distinctive class of proliferative lesions composed of cells with an aberrant accumulation of mitochondria (Tallini 1998). Tumors composed of oncotytic cells are particularly common among thyroid neoplasms of follicular cell derivation. Clinically, oncocytic thyroid tumors (also called Hurthle cell lesions) are more aggressive than their non-oncocytic counterparts, and are thus considered an adverse prognostic indicator. Possible genetic markers of such cancers have been described, however, to date there remains a need for new methods for accurate identification of cancers, in particular aggressive cancers such as oncocytic tumors.

### SUMMARY OF THE INVENTION

In a first embodiment, the invention provides a method of detecting cancer cells, (e.g., from suspected thyroid tumors) or cells having deregulated mitochondria in a subject comprising measuring a level of a lipid and/or metabolite (e.g., a cardiolipin) in a test sample from a subject and comparing the measured level to a reference level thereby generating a profile to detect the presence of cancer cells. In some aspects, the method is further defined as a method for detecting thyroid cancer in a subject. In particular aspects, the thyroid cancer is oncocytic thyroid cancer. In some aspects, the thyroid cancer is papillary thyroid cancer, follicular thyroid cancer, anaplastic thyroid cancer, or medullary thyroid cancer. In certain aspects, the method is further defined an ex vivo method. In some aspects, measuring a level of a lipid (e.g., cardiolipin) comprises performing mass spectroscopy on the sample.

In several aspects, the method comprises performing ambient ionization mass spectrometry (MS). For example, a method can involve preforming DESI-MSI. In further aspects, the method comprises performing 2D DESI-MSI. In certain aspects, performing 2D DESI-MSI comprises measuring a level of a lipid and/or metabolite in the sample. In specific aspects, the lipid is cardiolipin. In some aspects, the 2D DESI-MSI comprises a resolution of 500 um to 50 um, for example the resolution can be less than 500 um, 400 um, 300 um, 250 um, 200 um or 150 um.

In further aspects, the method further comprises comparing obtaining a reference profile and detecting the presence of cancer cells by comparing the profile from the sample to a reference profile. In particular aspects, the method further comprises measuring a level of a cardiolipin in a reference sample to obtain the reference level. In certain aspects, the test sample and the reference sample are obtained from the same subject or they may be obtained from different subjects. In particular aspects, the test sample is a sample from suspected tumor tissue and the reference sample is from normal tissue. In some specific aspects, the test sample and the reference sample are tissue samples wherein the test sample and the reference sample comprise two portions of the same tissue sample.

In further aspects, the level of a cardiolipin comprises a level of CL, ox-CL, CL+DG or CL+PC. In several aspects, the level of a cardiolipin is a level of one or more of the cardiolipins provided in Table 1 or 2. In certain specific aspects, the method further comprises measuring a level of a plurality of different cardiolipins in the sample. In additional aspects, the method further comprises measuring a level of at least 3, 4, 5, 6, 7, 8 or 9 different cardiolipins in the sample. In a particular aspect, the method comprises measuring a level of at least 10 different cardiolipins in the sample. In other aspects, said plurality of different cardiolipins are selected from those provided in Tables 1 or 2. In some aspects, measuring a level of a plurality of different cardiolipins in the sample is accomplished using DESI-MSI or 2D DESI-MSI. In some aspects, 2D DESI-MSI comprises a resolution of 500 μm to 50 μm. In specific aspects, the method comprises measuring a level of a plurality of ions corresponding to cardiolipins, wherein the plurality of ions are selected from those provided in Tables 1 or 2.

In still further aspects, the test sample is a tissue sample or a tissue section. In certain aspects, the test sample is an epidermal, thyroid, pancreatic, bladder, cervical, uterine, prostate, brain, kidney or liver tissue sample. In some aspects, the test sample may comprise suspected tumor tissue or a biopsy sample. In a particular aspect, the test sample is a thyroid tissue sample. In additional aspects, the sample is further subjected to histological staining. In further aspects, the method further comprises collecting the sample from the subject. In some aspects, collecting the sample comprises performing a biopsy. In specific aspects, collecting the sample comprises performing an ultrasound guided biopsy.

In yet another aspect, the method further comprises administering at least a first anticancer therapy to a subject identified as having a cancer. In some aspects, the anticancer therapy comprises radiation, immunotherapy, surgery or chemotherapy therapy. In a particular aspect, the cancer is a thyroid cancer and the anticancer therapy is an iodine based therapy.

In a further embodiment there is provided a method of treating a subject comprising selecting a patient determined to have a cancer in accordance with embodiments and aspects described above and administering at least a first anticancer therapy to the subject. In several aspects, the anticancer therapy comprises radiation, immunotherapy, surgery or chemotherapy therapy. In a particular aspect, the cancer is a thyroid cancer and the anticancer therapy is an iodine based therapy.

A further embodiment provides a method of detecting cells exhibiting mitochondrial dysregulation in a subject comprising measuring a level of a cardiolipin in a test sample from a subject and detecting the presence of cells exhibiting mitochondrial dysregulation based on the measured cardiolipin levels. In several aspects, the method is further defined an ex vivo method. In some aspects, the method further comprises comparing the measured level to a reference level to provide normalized level and detecting the presence of cells exhibiting mitochondrial dysregulation based on the normalized level. In additional aspects, the method if further defined as a method for detecting neurodegenerative disease, wherein the cells exhibiting mitochondrial dysregulation comprise neuronal cells. In certain aspects, the method is further defined as a method for detecting Barth Syndrome.

In further aspects, the method is further defined as a method for detecting cancer cells, wherein the cells exhibiting mitochondrial dysregulation comprise cancer cells. In some aspects, the method is further defined as a method for detecting oncocytic cancers in the subject. In particular aspects, measuring a level of a cardiolipin comprises performing mass spectroscopy on the sample. In specific aspects, the method comprises performing DESI-MSI, particularly 2D DESI-MSI. In certain aspects, 2D DESI-MSI comprises a resolution of 500 um to 50 um. In additional aspects, the method further comprises measuring a level of a cardiolipin in a reference sample to obtain the reference level.

In some aspects, the test sample and the reference sample are obtained from the same subject. In other aspects, the test sample and the reference sample are obtained from different subjects. In particular aspects, the test sample is a sample from suspected tumor tissue and the reference sample is from normal tissue. In other aspects, the test sample and the reference sample are tissue samples. In certain aspects, the test sample and the reference sample comprise two portions of the same tissue sample. In some aspects, the level of a cardiolipin comprises a level of ox-CL, CL+DG or CL+PC. In specific aspects, the level of a cardiolipin is a level of one or more of the cardiolipins provided in Table 1 or 2. In additional aspects, the method further comprises measuring a level of a plurality of different cardiolipins in the sample. In particular aspects, the method further comprises measuring a level of at least 3, 4, 5, 6, 7, 8 or 9 different cardiolipins in the sample. In some aspects, further comprising measuring a level of at least 10 different cardiolipins in the sample. In specific aspects, the plurality of different cardiolipins are selected from those provided in Tables 1 or 2.

In certain aspects, the method comprises measuring a level of a plurality of different cardiolipins in the sample using DESI-MSI. In some aspects, the method comprises measuring a level of a plurality of different cardiolipins in the sample using 2D DESI-MSI. In some aspects, 2D DESI-MSI comprises a resolution of 500 um to 50 um. In some aspects, the method comprises measuring a level of a plurality of ions corresponding to cardiolipins, wherein the plurality of ions are selected from those provided in Tables 1 or 2. In some aspects, the test sample is a tissue sample. In certain aspects, the test sample is a tissue section. In certain aspects, the test sample is an epidermal, thyroid, pancreatic, bladder, cervical, uterine, prostate, brain, kidney or liver tissue sample. In particular aspects, the test sample comprises suspected tumor tissue. In specific aspects, the test sample is a biopsy sample. In some aspects, the test sample is a kidney tissue sample. In a particular aspects, the test sample is a thyroid tissue sample. In some aspects, the sample is further subjected to histological staining.

In additional aspects, the method further comprises administering a therapy to a subject identified as having cells exhibiting mitochondrial dysregulation. In some aspects, the method further comprises administering at least a first anticancer therapy to a subject identified as having a cancer. In certain aspects, the anticancer therapy comprises radiation, immunotherapy, surgery or chemotherapy therapy. In some aspects, the cancer is a thyroid cancer and the anticancer therapy is an iodine based therapy.

In another embodiment, there is provided a method of treating a subject comprising selecting a patient determined to have cells exhibiting mitochondrial dysregulation in accordance with the embodiments and administering at least a first therapy to the subject. A further embodiment provides a method of treating a subject comprising selecting a patient determined to have a cancer in accordance with the embodiments, and administering at least a first anticancer therapy to the subject. In certain aspects, the anticancer therapy comprises radiation, immunotherapy, surgery or chemotherapy therapy. In particular aspects, the cancer is a thyroid cancer and the anticancer therapy is an iodine based therapy.

In still a further embodiment, the invention provides a method comprising measuring levels of a plurality of cardiolipins in a test sample from a subject using 2D DESI-MSI. In some aspects, 2D DESI-MSI comprises a resolution of 500 um to 50 um. In certain aspects, the method is further defined an ex vivo method. In some aspects, the tissue sample is a sample from suspected tumor tissue. In additional aspects, the levels of cardiolipins comprise level of CL, ox-CL, CL+DG and/or CL+PC. In some particular aspects, the levels of cardiolipins are selected from the cardiolipins provided in Table 1 or 2. In other aspects, the method further comprises measuring a level of at least 3, 4, 5, 6, 7, 8 or 9 different cardiolipins in the sample. In a specific aspect, the method comprises measuring a level of at least 10 different cardiolipins in the sample. In further aspects, the method comprises measuring a level of a plurality of ions corresponding to cardiolipins, wherein the plurality of ions are selected from those provided in Tables 1 or 2. In some aspects the test sample is a tissue sample or a tissue section. In certain aspects, the sample is an epidermal, thyroid, pancreatic, bladder, cervical, uterine, prostate, brain, kidney or liver tissue sample. In further particular aspects, the tissue sample is a biopsy sample or a thyroid tissue sample. In another aspect, the sample may be further subjected to histological staining.

In yet still a further embodiment, there is provided a tangible computer-readable medium comprising computer-readable code that, when executed by a computer, causes the computer to perform operations comprising receiving information corresponding a measurement of a cardiolipin level in a test sample and correlating the measured cardiolipin level of the test sample with a reference level, to produce a profile for the test sample. In certain aspects, the measurement of a cardiolipin level in the test sample comprises measurements of a plurality of cardiolipins. In several aspects, the plurality of cardiolipins are selected from those listed in Tables 1 and 2. In some aspects, the measurement of a cardiolipin level in the test sample may comprise a measurement of an ion generated by mass spectroscopy corresponding to the cardiolipin. In other aspects, the ion is selected from those of Tables 1 or 2.

In additional aspects, the tangible computer-readable medium further comprises receiving information corresponding a measurement of a cardiolipin level at a plurality of 2D positions in a test sample. In several aspects, the information corresponding a measurement of a cardiolipin level in a test sample comprises DESI-MSI data or 2D DESI-MSI data.

In another aspect, the tangible computer-readable medium further comprises analyzing the profile of the test sample to determine if the test sample is a cancer sample (e.g., an oncocytic cancer sample). In some aspects, the reference levels are stored in said tangible computer-readable medium. In certain aspects, receiving the information comprises receiving from a tangible data storage device information corresponding to the measurement of a cardiolipin level in a test sample. In additional aspects, the tangible computer-readable medium further comprises computer-readable code that, when executed by a computer, causes the computer to perform one or more additional operations comprising sending information corresponding the profile for the test sample to a tangible data storage device.

In still yet a further embodiment, the invention provides a tangible computer-readable medium comprising a computer-readable code comprising a database of values corresponding the levels of a plurality of cardiolipins levels in a biological sample and a computer-readable code that, when executed, selectively obtains the marker values from the database values and performs a calculation with the selectively obtained marker values.

In yet a further embodiment, there is provided a tangible computer-readable medium comprising a computer-readable code comprising a database of values corresponding the relative levels of a plurality of cardiolipins in a biological sample as compared to corresponding reference levels for the plurality of cardiolipins and a computer-readable code that, when executed, selectively obtains the marker values from the database values and performs a calculation with the selectively obtained marker values.

In certain aspects of the above embodiments, the obtained marker values provide information as to whether the biological sample comprised cancer cells. In some aspects, the plurality of cardiolipins are selected from those provided in Table 1 or 2.

As used herein in the specification and claims, “a” or “an” may mean one or more. As used herein in the specification and claims, when used in conjunction with the word “comprising”, the words “a” or “an” may mean one or more than one. As used herein, in the specification and claim, “another” or “a further” may mean at least a second or more.

As used herein in the specification and claims, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating certain embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-C. Representative negative ion mode DESI mass spectra of A) oncocytoma, B) non-oncocytoma and C) normal thyroid tissue.

FIGS. 2A-B. A) Workflow for imaging and mitochondria isolation experiments in thyroid tissues. B) DESI-MSI analysis of an oncocytic tumor, non-oncocytic tumor and normal thyroid tissues. The images on the left are from H&E stained tissues which were analyzed by non-destructive DESI-MSI. Scale bar=4 mm. Six representative images from different lipid ions, including PI (m/z 885.548), PS (m/z 788.544), PE (m/z 766.538), CL (m/z 738.502), CL (m/z 723.479) and FA (m/z 303.233) are presented.

FIGS. 3A-C. Mitochondria accumulation and changes in mitochondrial CL composition occur in oncocytic tumors. A) Confocal images for oncocytoma (O1 and O2), non-oncocytoma (NO1 and NO2), and normal (N1 and N2) tissues. B) UV-Vis analysis for protein concentration of mitochondrial isolation from three groups, including normal thyroid tissues (n=3), non-oncocytomas (n=3), and oncocytomas (n=4), (*P<0.001). C) Normalized CL intensities of the isolated mitochondria from normal thyroid tissues (n=3), non-oncocytomas (n=3), and oncocytomas (n=4), at the same concentration (3 μg protein/g of tissue) (**P<0.001). P<0.001 was considered as significant.

FIGS. 4A-C. SAM analysis for identifying statistically significant ions (m/z) from DESI-MSI data (n=30). A) Representative ions of ox-CLs, CLs, CL+DG and CL+PC found by SAM, B) Comparison of contrast values of CLs among oncocytoma, non-oncocytoma, and normal thyroid tissue, C) Comparison of contrast values of FAs among oncocytoma, non-oncocytoma, and normal thyroid tissue.

FIGS. 5A-C. Tandem mass spectrometry of three cardiolipins, A) ox-CL(18:2/18:2/18:2/9:1(OOH)), B) CL(20:2/18:2/18:1/16:2), and C) CL+PC (106:12).

FIG. 6. Analysis of mixture of CL (18:1/18:1/18:1:18:1) and PC (18:2/16:0) standards using DESI-MS. The inset shows the MS/MS of the ion at m/z 1106.2892 which was formed after mixing CL and PC together.

FIG. 7. DESI-MSI analysis of an oncocytic tumor, non-oncocytic tumor and normal thyroid tissues. The images on the left are from H&E stained tissues which were analyzed by non-destructive DESI-MSI. Scale bar=4 mm. Six representative images from different lipid ions, including PI (m/z 885.548), PS (m/z 788.544), PE (m/z 766.538), CL (m/z 738.502), CL (m/z 723.479) and FA (m/z 303.233) are presented.

FIG. 8. DESI-MS images of different CLs, ox-CLs, CL+DG and CL+PC from an oncocytic thyroid tissue.

FIG. 9. IHC staining images of Oncocytomas, Non-oncocytomas, and Normal tissues. Scale bar=4 mm.

FIGS. 10A-C. DESI-MS analysis of isolated mitochondria from A) oncocytoma, B) non-oncocytoma and C) normal tissue.

### DESCRIPTION OF ILLUSTRATIVE EMBODIMENTS

### I. THE PRESENT EMBODIMENTS

Studies detailed herein provide new methodologies for detecting cancer cells and, in particular, oncocytic tumor cells. In particular new lipid marker of the cancer cells were identified. In the studies herein mass spectroscopy, DESI-MS, was used to image and chemically characterize the lipid composition of thyroid tumors. The analysis revealed a novel molecular signature in oncocytic tumors characterized by an abnormally high abundance and chemical diversity of CL species. DESI-MS imaging and IHC experiments confirmed that the spatial distribution of these molecular ions overlapped with regions of accumulation of mitochondria-rich oncocytic cells. Moreover, fluorescence imaging confirmed that the oncocytic tumors investigated presented high accumulation of mitochondria when compared to non-oncocytic and normal thyroid tissue.

Using high-mass accuracy, high-mass resolution, and tandem MS experiments, 101 CL species directly from oncocytic thyroid tissues were identified. Amongst the CL species identified, 54 doubly charged molecular ions composed of CL bound to PC or DG were seen at high relative abundances in oncocytic tumors when compared to non-oncocytic or normal thyroid tissues. Likewise, 17 different ox-CL were identified in oncocytic tumors. Oxidization of other abundant polyunsaturated phospholipids were not observed in the studies, which indicates that this phenomena is primarily occurring for CL in oncocytic tumors themselves.

Thus, the studies herein provide new means for identification of cancer cells, such as oncocytic thyroid tumor cells, or cells having mitochondrial dysregulation by detecting abnormal expression and composition of CL and other lipids. In particular, MS detection of CL and CL oxidation products can be used to generate a profile indicating the presence of lesions in a patient. The presence of these profiles can then be used to guide patient therapy. For example, in the case of a patient identified to have an oncocytic tumor, a more aggressive therapy regime can be used to address the cancer. Thus, the methodologies and markers provided herein should provide a new avenue for accurate diagnosis and treatment for cancers, such as thyroid cancers.

### II. ASSAY METHODOLOGIES

In some aspects, the present disclosure provides methods of determining the presence of a tumor by identifying specific patterns of lipids such as cardiolipins. These patterns may be determined by measuring the presence of specific lipid ions using mass spectroscopy. Some non-limiting examples of ionizations methods include chemical ionization, atmospheric-pressure chemical ionization, electron ionization, fast atom bombardment, electrospray ionization, and matrix-assisted laser desorption/ionization. Additional ionization methods include inductively coupled plasma sources, photoionization, glow discharge, field desorption, thermospray, desorption/ionization on silicon, direct analysis in real time, secondary ion mass spectroscopy, spark ionization, and thermal ionization.

In particular, the present methods may be applied to an ambient ionization source or method for obtaining the mass spectral data such as extraction ambient ionization source. Extraction ambient ionization sources are methods with a solid or liquid extraction processes dynamically followed by ionization. Some non-limiting examples of extraction ambient ionization sources include air flow-assisted desorption electrospray ionization (AFADESI), direct analysis in real time (DART), desorption electrospray ionization (DESI), desorption ionization by charge exchange (DICE), electrode-assisted desorption electrospray ionization (EADESI), electrospray laser desorption ionization (ELDI), electrostatic spray ionization (ESTASI), Jet desorption electrospray ionization (JeDI), laser assisted desorption electrospray ionization (LADESI), laser desorption electrospray ionization (LDESI), matrix-assisted laser desorption electrospray ionization (MALDESI), nanospray desorption electrospray ionization (nano-DESI), or transmission mode desorption electrospray ionization (TM-DESI). In some embodiments, the ionization source used in the methods described herein is desorption electrospray ionization.

DESI is an ionization technique used to prepare a mass spectra of organic molecules or biomolecules. The ionization technique is an ambient ionization technique which uses atmospheric pressure in the open air and under ambient conditions. DESI is an ionization technique which combines two other ionization techniques: electrospray ionization as well as desorption ionization. Ionization is affected by directing electrically charged droplets at the surface that is millimeters away from the electrospray source. The electrospray mist is then pneumatically directed at the sample. Resultant droplets are desorbed and collected by the inlet into the mass spectrometer. These resultant droplets contain additional analytes which have been desorbed and ionized from the surface. These analytes travel through the air at atmospheric pressure into the mass spectrometer for determination of mass and charge. One of the hallmarks of DESI is the ability to achieve ambient ionization without substantial sample preparation.

As with many mass spectroscopy methods, ionization efficiency can be optimized by modifying the spray conditions such as the solvent sprayed, the pH, the gas flow rates, the applied voltage, and other aspects which affect ionization of the sprayed solution. In particular, the present methods contemplate the use of a solvent or solution which is compatible with human issue. Some non-limiting examples of solvent which may be used as the ionization solvent include water, methanol, acetonitrile, dimethylformamide, an acid, or a mixture thereof. In some embodiments, the method contemplates a mixture of acetonitrile and dimethylformamide. The amounts of acetonitrile and dimethylformamide may be varied to enhance the extraction of the analytes from the sample as well as increase the ionization and volatility of the sample. In some embodiments, the composition contains from about 5:1 (v/v) dimethylformamide:acetonitrile to about 1:5 (v/v) dimethyl [symbol] formamide:acetonitrile such as 1:1 (v/v) dimethylformamide: acetonitrile.

Additionally, two useful parameters are the impact angle of the spray and the distance from the spray tip to the surface. Generally, the electrospray tip is placed from about 0.1-25 mm from the surface especially from 1-10 mm. In some embodiments, a placement from about 3-8 mm is useful for a wide range of different application such as those described herein. Additionally, varying the angle of the tip to the surface (known as the incident angle or a) may be used to optimize the ionization efficacy. In some embodiments, the incident angle may be from about 0° to about 90°. In some aspects, a poorly ionizing analytes such as a biomolecule will have a larger incident angle while better ionizing analytes such as low molecular weight biomolecules and organic compounds have smaller incident angle. Without wishing to be bound by any theory, it is believed that the differences in the incident angle results from the two different ionization mechanisms for each type of molecule. The poorly ionizing biomacromolecules may be desorbed by the droplet where multiple charges in the droplet may be transferred to the biomacromolecule. On the other hand, low molecular weight molecules may undergo charge transfer as either a proton or an electron. This charge transfer may be from a solvent ion to an analyte on the surface, from a gas phase solvent ion to an analyte on the surface, or from a gas phase solvent ion to a gas phase analyte molecule.

Additionally, the collection efficiency or the amount of desorbed analyte collected by the collector can be optimized by varying the collection distance from the inlet of the mass spectrometer and the surface as well as varying the collection angle (β). In general, the collection distance is relatively short from about 0 mm to about 5 mm. In some cases, the collection distance may be from about 0 mm to about 2 mm. Additionally, the collection angle (β) is also relatively small from about 1° to about 30° such as from 5° to 10°.

Each of these components may be individually adjusted to obtain an sufficient ionization and collection efficiencies. Within the DESI source, the sample may be placed on a 3D moving stage which allows precise and individual control over the ionization distance, the collection distance, the incident angle, and the collection angle.

Finally, the mass spectrometer may use a variety of different mass analyzers. Some non-limiting examples of different mass analyzers include time-of-flight, quadrupole mass filter, ion trap such as a 3D quadrupole ion trap, cylindrical ion trap, linear quadrupole ion trap, or an orbitrap, or a fourier transform ion cyclotron resonance device.

### III. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Molecular Characterization of Cardiolipins in Oncocytic Tissue

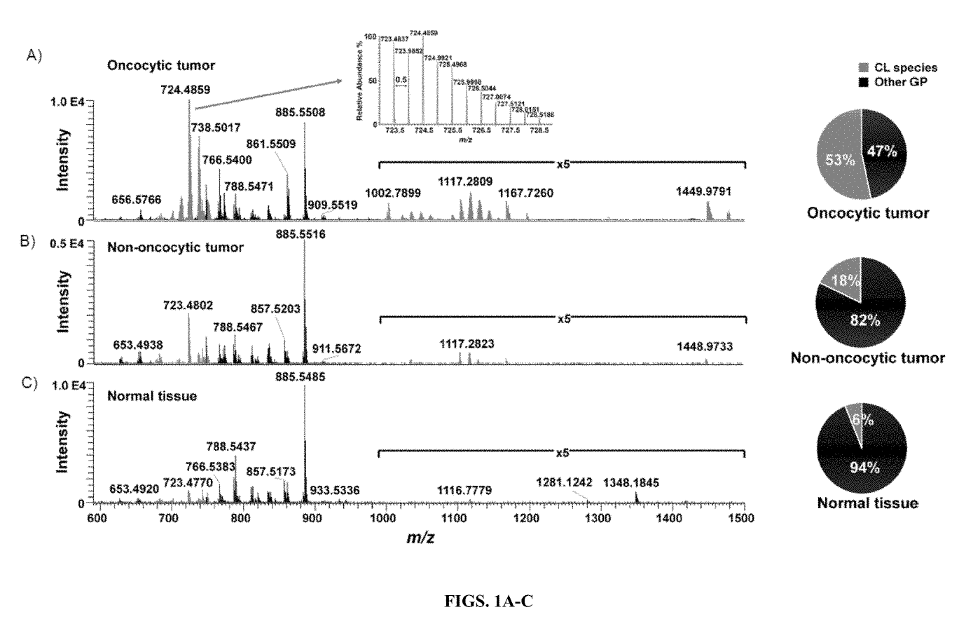

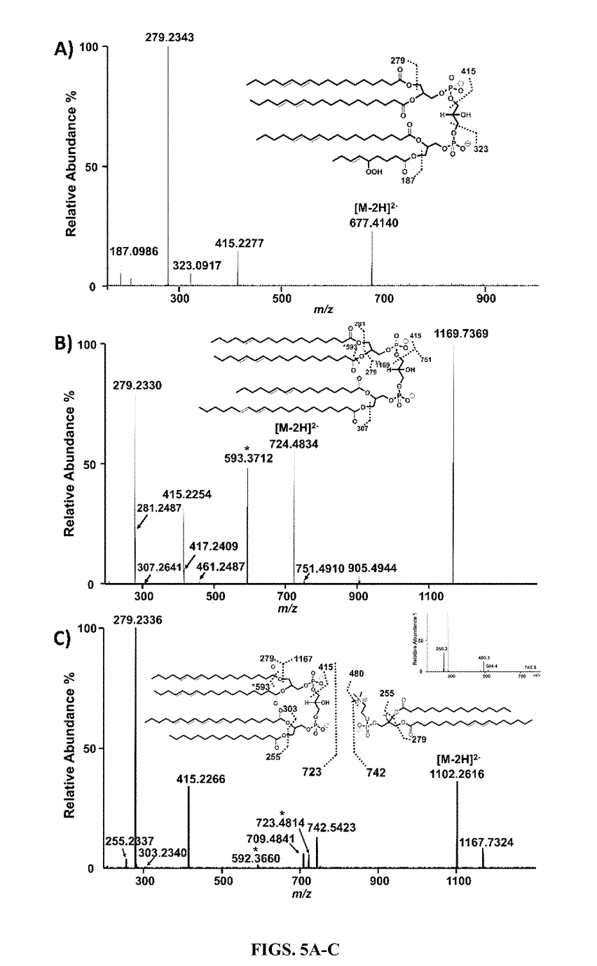

Negative ion mode DESI-MS was used to analyze 30 human thyroid samples, including 10 oncocytic thyroid tumors (8 hurthle cell adenomas and 2 hurthle cell carcinomas), 10 non-oncocytic thyroid tumors (5 papillary thyroid carcinoma and 5 follicular thyroid adenoma) and 10 normal thyroid tissues. The mass spectra obtained presented high relative abundances of several molecular ions commonly characterized as lipid species in the negative ion mode DESI mass spectra of human tissues, including fatty acids (m/z 200-400), and glycerophospholipids (GP) (m/z 700-1000) such as glycerophosphoinositols (PI), glycerophosphoethanolamine (PE), and glycerophosphoserines (PS). Normal thyroid tissue displayed high relative abundances of PI (20:4/18:0) (m/z 885.548), PS (20:3/18:0) (m/z 812.544), PS (18:1/18:0) (m/z 788.544), PE (20:4/18:0) (m/z 766.538), PE (18:2/18:1) (m/z 742.538) and phosphatidic acids (PA) (18:1/18:0) (m/z 701.512) (FIG. 1C) which are lipid ions commonly detected from mammalian tissues. In contrast, the mass spectra obtained from oncocytic tumor samples showed a very distinct and reproducible profile with abnormally high relative abundances of a series of doubly charged ions in the mass range from m/z 590-760, and m/z 1000-1200 (FIG. 1A). The spectra was remarkably rich in molecular diversity, and unlike what commonly observed in human cancer tissues by DESI-MS imaging. Using high mass accuracy measurements and tandem MS analysis, we identified these doubly charged ions as CL species. CL have been previously investigated by electrospray ionization and tandem MS approaches and present key fragment ions that enable structural characterization. (19, 20) For example, tandem MS experiments of doubly charged molecular ion m/z 724.483 yielded fragment ions corresponding to 18:2-carboxylate anion (m/z 279.233), 18:1-carboxylate anion (m/z 281.249), 20:2-carboxylate anion (m/z 307.264), lyso-PA fragments (m/z 415.225, m/z 417.241 and m/z 461.249), a doubly charged ketene (m/z 593.371) arising from loss of the 18:2-fatty acyl substituent, and a fragment ion at m/z 1169.737 produced by neutral loss of FA(18:2), indicating that the molecular ions corresponds to CL(20:2/18:2/18:1/16:2) (FIGS. 5A-C). High mass accuracy measurements agrees with the exact mass (m/z 724.4867) of proposed molecular formula (C₈₁H₁₄₄O₁₇P₂) with a mass error of −1.7 ppm. Note that isomerism of the double bonds in the fatty acid (FA) chains of GP complicates precise structural assignment, which is why acyl chains are only tentatively assigned. Furthermore, several combinations of the four acyl chains at different positions in the CL structure are possible, thus, the exact configuration cannot be assigned by the method. In total, 31 CL molecular ions were identified and characterized, except for two CL ions which present insufficient fragment ions intensity. The singly charged CL molecular ions were also observed from m/z 1400-1500 at high relative intensities in the oncocytic tumor when compared to non-oncocytic and normal thyroid tissues.

Several ox-CL species were also identified in the oncocytic tumors mass spectra. For example, tandem MS experiments of doubly charged molecular ion m/z 677.414 yielded oxidized carboxylate anion (9:1-OOH) (m/z 187.099), 18:2-carboxylate anion (m/z 279.234), oxidized lyso-PA from (9:1-OOH) at m/z 323.092, and lyso-PA at m/z 415.228, indicating that the CL molecular species is ox-CL(18:2/18:2/18:2/9:1(OOH)) (FIGS. 5A-C). (21) Altogether, 17 different ox-CLs were identified in oncocytic tumors. Note that these oxidized species were also observed in oncocytic tissues when no voltage was applied in the DESI source. (22) Interestingly, this oxidation effect was specific to CL, as other polyunsaturated GP at similar relative abundances were not detected in their oxidized forms by our method. Thus, the data indicates that the detected ox-CL are not an effect of the experimental conditions used but rather endogenous molecules present in oncocytic tissues.

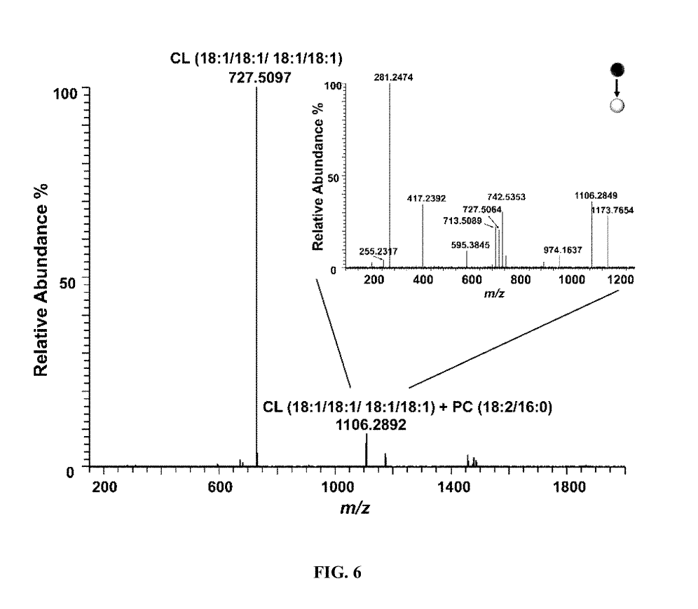

An uncommon series of doubly charged peaks from m/z 1000-1200 were observed in high relative abundance in oncocytic tumors when compared to non-oncocytic and normal thyroid tissues. These peaks were identified using a series of tandem MS experiments as a combination of CL with diacylglycerides (DG) (m/z 1000-1100), or, more predominantly observed, glycerophosphocholines (PC) (m/z 1100-1200). The ion m/z 1102.262, for example, was identified as CL+PC (106:12) using MS² and MS³ experiments (FIGS. 5A-C). The chemical structure of many fragment ions include structural components of both PC and CL molecules, which indicates that these species are strongly bound, through what was hypothesized to be an ionic bond within a concatenated structure. To confirm the chemical composition of these ions, DESI-MS analysis was performed on a mixture of CL and PC standards, and the formation of these doubly charged species was observed which presented identical fragmentation patterns (FIG. 6) to those observed in tissue. PC were not observed in negative ion mode in the experiments, thus, it was interesting to observe these molecules bound to CL species.

In total, 101 different CL species including CL (singly and doubly charged), ox-CL, CL+PC, and CL+DG were identified in oncocytic tumors and are described in Table 1 and Table 2 (note that only C¹² isotope was included). In contrast to oncocytic follicular thyroid tumors, the mass spectra obtained from non-oncocytic follicular thyroid tumors showed high relative abundances of PI (20:4/17:0) at m/z 871.536, PI (20:4/16:0) at m/z 857.520, PI (18:2/16:0) at m/z 833.518, and PE (18:1/O-16:1) at m/z 700.530 (FIG. 1B). CL species were also observed but at lower relative intensities than what observed in oncocytic follicular tumors, while oxidized species were undetectable using our method. Non-oncocytic papillary thyroid tumors presented different molecular profiles amongst samples. In 3 out of the 5 non-oncocytic papillary thyroid tumors investigated, the relative abundances of CL species were higher when compared to non-oncocytic follicular thyroid tumors, but consistently lower and less diverse to what observed in oncocytic tumors.

TABLE 1
CL and ox-CL species identified using high mass resolution/high mass accuracy and tandem
mass spectrometry analyses.

| | | | | Mass | |
|---|---|---|---|---|---|
| Measured | Lipid | Tentative | Exact | Error | Proposed |
| m/z | Class^{[a]} | Attribution | m/z | (ppm)^{[c]} | Formula |
| 592.3641 | CL | CL(54:5) | 592.3640 | 0.2 | C₆₃H₁₁₂O₁₆P₂ |
| 593.3722 | CL | CL(54:4) | 593.3718 | 0.7 | C₆₃H₁₁₄O₁₆P₂ |
| 669.4135 | ox-CL^{[b]} | 20:4/18:2/16:0/9:1(OH) | 669.4137 | −0.2 | C₇₂H₁₂₆O₁₈P₂ |
| 670.4215 | ox-CL | 18:2/18:2/18:1/9:1(OH) | 670.4215 | −0.1 | C₇₂H₁₂₈O₁₈P₂ |
| 677.4108 | ox-CL | 18:2/18:2/18:2/9:1(OOH) | 677.4112 | −0.1 | C₇₂H₁₂₆O₁₉P₂ |
| 678.4187 | ox-CL | 18:2/18:2/18:1/9:1(OOH) | 678.4190 | −0.3 | C₇₂H₁₂₈O₁₉P₂ |
| 689.4292 | ox-CL | 18:2/18:2/18:2/12:2(OH) | 689.4293 | −0.2 | C₇₅H₁₃₀O₁₈P₂ |
| 690.4352 | ox-CL | 18:2/18:2/18:1/12:2(OH) | 690.4372 | −2.8 | C₇₅H₁₃₂O₁₈P₂ |
| | | 20:4/18:1/16:0/12:2(OH) | | | |
| 691.4261 | ox-CL | 20:2/18:2/16:0/12:2(OOH) | 691.4268 | −1.0 | C₇₄H₁₃₀O₁₉P₂ |
| 697.4279 | ox-CL | CL(OO-65:8) | 697.4268 | 1.1 | C₇₅H₁₃₀O₁₉P₂ |
| 697.4635 | CL | 18:2/18:2/18:2/14:0 | 697.4632 | 0.5 | C₇₇H₁₃₈O₁₇P₂ |
| | | 20:2/18:2/16:2/14:0 | | | |
| 698.4355 | ox-CL | CL(OO-65:7) | 698.4346 | 0.9 | C₇₅H₁₃₂O₁₉P₂ |
| 698.4709 | CL | 18:2/18:2/18:1/14:0 | 698.4710 | −0.2 | C₇₇H₁₄₀O₁₇P₂ |
| 699.4437 | ox-CL | CL(OO-65:6) | 699.4425 | 1.2 | C₇₅H₁₃₄O₁₉P₂ |
| 699.4774 | CL | 18:2/18:2/18:0/14:0 | 699.4788 | −2.0 | C₇₇H₁₄₂O₁₇P₂ |
| 700.4866 | CL | 18:1/18:1/18:1/14:0 | 700.4867 | −0.1 | C₇₇H₁₄₄O₁₇P₂ |
| 701.4929 | CL | 18:1/18:1/18:0/14:0 | 701.4945 | −0.5 | C₇₇H₁₄₆O₁₇P₂ |
| 706.4869 | CL | 18:2/18:1/18:1/15:0 | 706.4867 | 0.3 | C₇₈H₁₄₄O₁₇P₂ |
| 710.4709 | CL | 18:2/18:2/18:2/16:1 | 710.4710 | 0.1 | C₇₉H₁₄₀O₁₇P₂ |
| 711.4767 | CL | 18:2/18:2/18:1/16:1 | 711.4788 | 0.4 | C₇₉H₁₄₂O₁₇P₂ |
| | | 18:2/18:2/18:2/16:0 | | | |
| 712.4849 | CL | 18:2/18:2/18:1/16:0 | 712.4867 | 0.5 | C₇₉H₁₄₄O₁₇P₂ |
| | | 18:2/18:1/18:1/16:1 | | | |
| 713.4927 | CL | 18:2/18:1/18:1/16:0 | 713.4945 | 0.6 | C₇₉H₁₄₆O₁₇P₂ |
| 714.5012 | CL | 18:1/18:1/18:1/16:0 | 714.5023 | 0.4 | C₇₉H₁₄₈O₁₇P₂ |
| 722.4711 | CL | 20:4/18:3/18:1/16:1 | 722.4710 | 0.1 | C₈₁H₁₄₀O₁₇P₂ |
| 723.4789 | CL | 20:4/18:2/18:2/16:0 | 723.4788 | 0.1 | C₈₁H₁₄₂O₁₇P₂ |
| 724.4851 | CL | 20:2/18:2/18:1/16:2 | 724.4867 | −1.7 | C₈₁H₁₄₄O₁₇P₂ |
| 725.4936 | CL | 20:3/18:2/18:1/16:0 | 725.4945 | −0.9 | C₈₁H₁₄₆O₁₇P₂ |
| | | 20:2/18:2/18:1/16:1 | | | |
| 726.5015 | CL | 20:2/18:2/18:1/16:0 | 726.5023 | −0.8 | C₈₁H₁₄₈O₁₇P₂ |
| 727.5097 | CL | 20:2/18:2/18:0/16:0 | 727.5101 | −0.5 | C₈₁H₁₅₀O₁₇P₂ |
| | | 20:2/18:1/18:1/16:0 | | | |
| 730.4684 | ox-CL | CL(O72:9) | 730.4685 | −0.2 | C₈₁H₁₃₈O₁₈P₂ |
| 731.4768 | ox-CL | CL(O72:8) | 730.4763 | 0.8 | C₈₁H₁₄₀O₁₈P₂ |
| 732.4821 | ox-CL | 18:2/18:1/19:1/17:3(OH) | 730.4841 | −2.8 | C₈₁H₁₄₂O₁₈P₂ |
| | | 18:4(OH)/18:2/18:1/16:0 | | | |
| 735.4783 | CL | 20:4/18:2/18:2/18:2 | 735.4788 | −0.7 | C₈₃H₁₄₂O₁₇P₂ |
| 736.4866 | CL | 20:4/18:2/18:2/18:1 | 736.4867 | −0.1 | C₈₃H₁₄₄O₁₇P₂ |
| | | 20:3/18:2/18:2/18:2 | | | |
| 737.4944 | CL | 20:4/18:2/18:1/18:1 | 737.4945 | −0.1 | C₈₃H₁₄₆O₁₇P₂ |
| | | 20:3/18:2/18:2/18:1 | | | |
| | | 20:2/18:2/18:2/18:2 | | | |
| 738.5022 | CL | 20:4/20:2/18:1/16:0 | 738.5023 | −0.2 | C₈₃H₁₄₈O₁₇P₂ |
| | | 20:3/18:2/18:1/18:1 | | | |
| | | 20:2/18:2/18:2/18:1 | | | |
| 739.4740 | ox-CL | CL(OO72:8) | 739.4738 | 0.2 | C₈₁H₁₄₂O₁₉P₂ |
| 740.4803 | ox-CL | CL(OO72:7) | 740.4810 | −1.3 | C₈₁H₁₄₄O₁₉P₂ |
| 745.4914 | ox-CL | CL(O74:8) | 745.4910 | −0.7 | C₈₃H₁₄₄O₁₈P₂ |
| 746.4982 | ox-CL | CL(O74:7) | 746.4998 | −2.1 | C₈₃H₁₄₆O₁₈P₂ |
| 747.4780 | CL | 22:6/20:4/18:2/16:0 | 747.4788 | −0.8 | C₈₅H₁₄₂O₁₇P₂ |
| 748.4836 | CL | 22:6/20:4/18:1/16:0 | 748.4867 | 0.1 | C₈₅H₁₄₄O₁₇P₂ |
| 749.4942 | CL | 22:5/20:4/18:1/16:0 | 749.4945 | −0.4 | C₈₅H₁₄₆O₁₇P₂ |
| 750.5024 | CL | 22:4/20:4/18:1/16:0 | 750.5023 | −1.1 | C₈₅H₁₄₈O₁₇P₂ |
| 751.5101 | CL | 22:4/20:4/18:0/16:0 | 751.5101 | 0.1 | C₈₅H₁₅₀O₁₇P₂ |
| 752.5172 | CL | 22:4/20:4/18:0/16:0 | 752.5180 | −0.9 | C₈₅H₁₅₂O₁₇P₂ |
| 753.5263 | CL | 22:4/20:3/18:0/16:0 | 752.5258 | 0.7 | C₈₅H₁₅₄O₁₇P₂ |

^{[a]}CL = cardiolipin (X:Y) denotes the total number of carbons and double bonds in the fatty acid chains.
^{[b]}ox-CL = oxidized cardiolipin
^{[c]}Mass errors were calculated based on the exact monoisotopic m/z of the deprotonated form of the assigned molecules.

TABLE 2
CL + DG and CL + PC species identified using high mass resolution/high mass accuracy
and tandem mass spectrometry analyses.

| | | | | Mass | |
|---|---|---|---|---|---|
| Measured | Lipid | Tentative | Exact | Error | Proposed |
| m/z | Class | Attribution | m/z | (ppm) | Formula |
| 1019.7316 | CL + DG | CL + DG(106:10) | 1019.7322 | −0.6 | C₁₁₈H₂₁₀O₂₂P₂ |
| 1020.7387 | CL + DG | CL + DG(106:9) | 1020.7400 | −1.3 | C₁₁₈H₂₁₂O₂₂P₂ |
| 1021.7440 | CL + DG | CL + DG(106:8) | 1021.7478 | −3.7 | C₁₁₈H₂₁₄O₂₂P₂ |
| 1022.7525 | CL + DG | CL + DG(106:7) | 1022.7556 | −3.0 | C₁₁₈H₂₁₆O₂₂P₂ |
| 1031.7322 | CL + DG | CL + DG(108:12) | 1031.7322 | <0.1 | C₁₂₀H₂₁₀O₂₂P₂ |
| 1032.7390 | CL + DG | CL + DG(108:11) | 1032.7410 | −1.9 | C₁₂₀H₂₁₂O₂₂P₂ |
| 1033.7461 | CL + DG | CL + DG(108:10) | 1033.7478 | −1.6 | C₁₂₀H₂₁₄O₂₂P₂ |
| 1034.7526 | CL + DG | CL + DG(108:9) | 1034.7556 | −2.9 | C₁₂₀H₂₁₆O₂₂P₂ |
| 1035.7604 | CL + DG | CL + DG(108:8) | 1035.7635 | −3.0 | C₁₂₀H₂₁₈O₂₂P₂ |
| 1036.7675 | CL + DG | CL + DG(108:7) | 1036.7713 | −3.7 | C₁₂₀H₂₂₀O₂₂P₂ |
| 1044.7405 | CL + DG | CL + DG(110:13) | 1044.7465 | −5.7 | C₁₂₂H₂₁₄O₂₂P₂ |
| 1045.7477 | CL + DG | CL + DG(110:12) | 1045.7478 | −0.1 | C₁₂₂H₂₁₆O₂₂P₂ |
| 1046.7529 | CL + DG | CL + DG(110:11) | 1046.7556 | −2.6 | C₁₂₂H₂₁₈O₂₂P₂ |
| 1047.7614 | CL + DG | CL + DG(110:10) | 1047.7635 | −2.0 | C₁₂₂H₂₂₀O₂₂P₂ |
| 1048.7702 | CL + DG | CL + DG(110:9) | 1048.7713 | −1.0 | C₁₂₂H₂₂₂O₂₂P₂ |
| 1049.7748 | CL + DG | CL + DG(110:8) | 1049.7791 | −4.1 | C₁₂₂H₂₂₄O₂₂P₂ |
| 1057.7463 | CL + DG | CL + DG(112:14) | 1057.7478 | −1.4 | C₁₂₄H₂₁₄O₂₂P₂ |
| 1058.7523 | CL + DG | CL + DG(112:13) | 1058.7556 | −3.1 | C₁₂₄H₂₁₆O₂₂P₂ |
| 1059.7627 | CL + DG | CL + DG(112:12) | 1059.7635 | −0.8 | C₁₂₄H₂₁₈O₂₂P₂ |
| 1060.7675 | CL + DG | CL + DG(112:11) | 1060.7713 | −3.6 | C₁₂₄H₂₂₀O₂₂P₂ |
| 1061.7766 | CL + DG | CL + DG(112:10) | 1061.7791 | −2.4 | C₁₂₄H₂₂₂O₂₂P₂ |
| 1062.7833 | CL + DG | CL + DG(112:9) | 1062.7869 | −3.4 | C₁₂₄H₂₂₄O₂₂P₂ |
| 1071.7613 | CL + DG | CL + DG(114:14) | 1071.7635 | −2.1 | C₁₂₆H₂₁₈O₂₂P₂ |
| 1072.7684 | CL + DG | CL + DG(114:13) | 1072.7713 | −2.7 | C₁₂₆H₂₂₀O₂₂P₂ |
| 1073.7771 | CL + DG | CL + DG(114:12) | 1073.7863 | −8.6 | C₁₂₆H₂₂₂O₂₂P₂ |
| 1074.7843 | CL + DG | CL + DG(114:11) | 1074.7869 | −2.4 | C₁₂₆H₂₂₄O₂₂P₂ |
| 1075.7928 | CL + DG | CL + DG(114:10) | 1075.7948 | −1.9 | C₁₂₆H₂₂₆O₂₂P₂ |
| 1089.2522 | CL + PC | CL + PC(104:11) | 1089.2521 | 0.1 | C₁₂₁H₂₂₀O₂₅NP₃ |
| 1090.2583 | CL + PC | CL + PC(104:10) | 1090.2599 | −1.5 | C₁₂₁H₂₂₂O₂₅NP₃ |
| 1091.2647 | CL + PC | CL + PC(104:9) | 1091.2677 | −2.7 | C₁₂₁H₂₂₄O₂₅NP₃ |
| 1092.2732 | CL + PC | CL + PC(104:8) | 1092.2756 | −2.2 | C₁₂₁H₂₂₆O₂₅NP₃ |
| 1093.2802 | CL + PC | CL + PC(104:7) | 1093.2834 | −2.9 | C₁₂₁H₂₂₈O₂₅NP₃ |
| 1094.2873 | CL + PC | CL + PC(104:8) | 1094.2912 | −3.6 | C₁₂₁H₂₃₀O₂₅NP₃ |
| 1102.2593 | CL + PC | CL + PC(106:12) | 1102.2599 | −0.5 | C₁₂₃H₂₂₂O₂₅NP₃ |
| 1103.2670 | CL + PC | CL + PC(106:11) | 1103.2677 | −0.6 | C₁₂₅H₂₂₄O₂₅NP₃ |
| 1104.2745 | CL + PC | CL + PC(106:10) | 1104.2756 | −1.0 | C₁₂₅H₂₂₄O₂₅NP₃ |
| 1105.2811 | CL + PC | CL + PC(106:9) | 1105.2834 | −2.1 | C₁₂₅H₂₂₄O₂₅NP₃ |
| 1115.2657 | CL + PC | CL + PC(108:11) | 1115.2677 | −1.8 | C₁₂₅H₂₂₄O₂₅NP₃ |
| 1116.2740 | CL + PC | CL + PC(108:10) | 1116.2756 | −1.4 | C₁₂₅H₂₂₆O₂₅NP₃ |
| 1117.2816 | CL + PC | CL + PC(108:9) | 1117.2834 | −1.6 | C₁₂₅H₂₂₈O₂₅NP₃ |
| 1118.2878 | CL + PC | CL + PC(108:8) | 1118.2912 | −3.0 | C₁₂₅H₂₃₀O₂₅NP₃ |
| 1119.2960 | CL + PC | CL + PC(108:7) | 1119.2990 | −2.7 | C₁₂₅H₂₃₂O₂₅NP₃ |
| 1128.2744 | CL + PC | CL + PC(110:14) | 1128.2756 | −1.1 | C₁₂₇H₂₂₆O₂₅NP₃ |
| 1129.2818 | CL + PC | CL + PC(110:13) | 1129.2834 | −1.4 | C₁₂₇H₂₂₈O₂₅NP₃ |
| 1130.2880 | CL + PC | CL + PC(110:12) | 1130.2912 | −2.8 | C₁₂₇H₂₃₀O₂₅NP₃ |
| 1131.2939 | CL + PC | CL + PC(110:11) | 1131.2990 | −4.5 | C₁₂₇H₂₃₂O₂₅NP₃ |
| 1132.3024 | CL + PC | CL + PC(110:10) | 1132.3069 | −4.0 | C₁₂₇H₂₃₄O₂₅NP₃ |
| 1133.3098 | CL + PC | CL + PC(110:9) | 1133.3147 | −4.3 | C₁₂₇H₂₃₆O₂₅NP₃ |
| 1141.2814 | CL + PC | CL + PC(112:13) | 1141.2834 | −1.8 | C₁₂₉H₂₂₈O₂₅NP₃ |
| 1142.2887 | CL + PC | CL + PC(112:12) | 1142.2912 | −2.2 | C₁₂₉H₂₃₀O₂₅NP₃ |
| 1143.2960 | CL + PC | CL + PC(112:11) | 1143.2990 | −2.6 | C₁₂₉H₂₃₂O₂₅NP₃ |
| 1144.3048 | CL + PC | CL + PC(112:10) | 1144.3069 | −1.8 | C₁₂₉H₂₃₄O₂₅NP₃ |
| 1145.3104 | CL + PC | CL + PC(112:9) | 1145.3147 | −3.8 | C₁₂₉H₂₃₆O₂₅NP₃ |
| 1146.3199 | CL + PC | CL + PC(112:8) | 1146.3225 | −2.3 | C₁₂₉H₂₃₈O₂₅NP₃ |

Evident changes in the relative abundances of FAs were also observed within the three thyroid tissue groups analyzed. Overall, oncocytic and non-oncocytic tumors showed higher total and relative abundances of FAs when compared to what were observed in normal thyroid tissues. Amongst the tumor samples, high relative abundances of FAs of longer carbon chains were observed in the oncocytic tumor tissues, such as 20:3-carboxylate anion (m/z 305.249), 20:2-carboxylate anion (m/z 307.265), 22:5-carboxylate anion (m/z 329.249), and 22:5-carboxylate anion (m/z 337.312), while FAs with shorter carbon chains were seen at higher relative intensities in non-oncocytic tumors, including 16:1-carboxylate anion (m/z 253.218), 18:3-carboxylate anion (m/z 277.218) and 18:0-carboxylate anion (m/z 283.265).

### Example 2

### Cardiolipin Distribution Correlates with Oncocytic Cells and Mitochondria Accumulation in Tissues

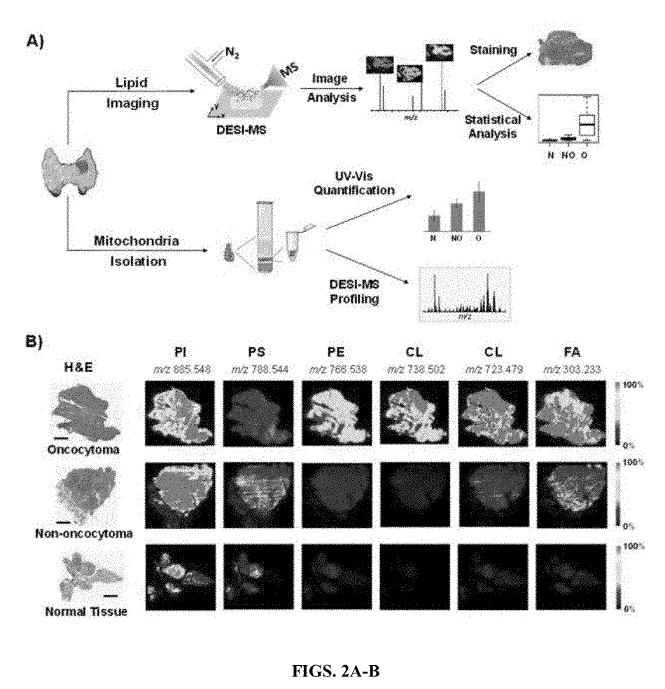

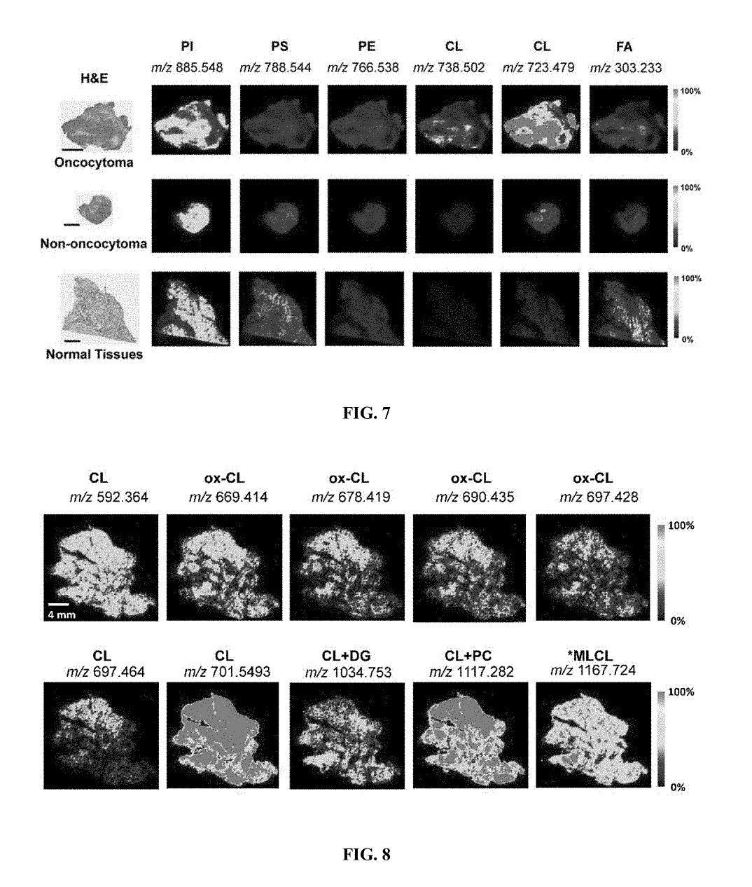

2D DESI-MSI experiments were performed to examine the spatial distribution of the molecular ions detected from thyroid tissues (FIG. 2A). Of particular interest was whether the spatial distribution of CL ions co-localized with specific histological features in oncocytic tumor tissues. FIG. 2B shows the DESI-MS images obtained for selected molecular ions for an oncocytic sample, a non-oncocytic sample, and normal thyroid tissue sample (additional imaging results are shown in FIG. 7). Optical images of the same tissue section which were H&E stained after DESI-MSI are also presented. All oncocytic tumors analyzed showed characteristic histological features with enlarged cells of high cytoplasmatic volume that accommodates the increased number of mitochondria. In many samples, regions predominantly composed of cancer cells were observed adjacent or within regions defined regions of fibrosis tissues. In oncocytic tumors, the molecular distribution of CL species was co-localized, homogeneous, and remarkably high within the regions with oncocytic tumor cells and were in lower intensities in fibrosis regions in sample, as shown for m/z 738.502 and m/z 723.479 (FIG. 2B). Similar spatial distribution was observed for other CL, ox-CL, CL+PC, and CL+DG molecular ions (FIG. 8). In all normal thyroid tissue analyzed, common patterns of the cellular organization were observed, with spherical follicles surrounded by a single layer of follicular cells and scattered parafollicular cells. The molecular images obtained for normal tissues showed lipid signal co-localized with follicular cells (FIG. 2B). Follicles did not show lipid profiles and are thus seen as dark regions in the DESI-MS ion images. Non-oncocytic follicular and papillary thyroid tumors showed typical histological patterns, and displayed a homogenous molecular distribution of the most abundant molecular ions within regions of tumor cell.

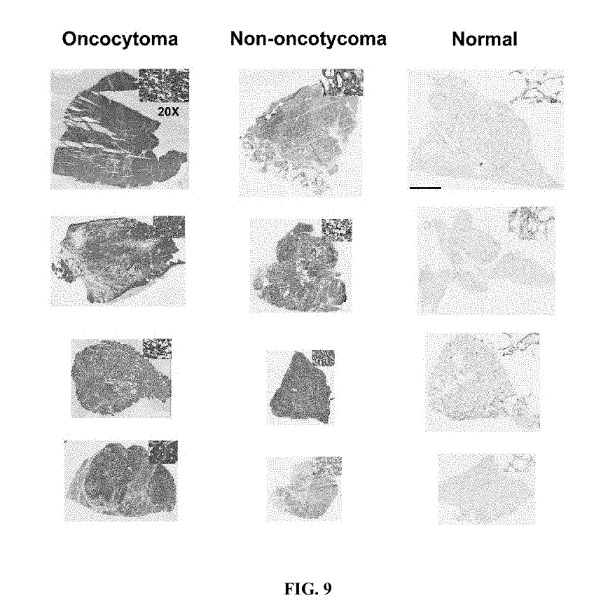

To evaluate if CL distribution correlates with regions of mitochondrial accumulation in oncocytic tumors, immunohistochemistry (IHC) was performed with an anti-mitochondrial antibody in tissues sections adjacent to those imaged by DESI-MS. Positive staining for mitochondria was observed for all thyroid tumors analyzed, while negative (weak) staining was observed for all normal thyroid tissues. As expected, strong mitochondrial staining for all oncocytic tissues was observed (FIG. 9). Spatial agreement was observed between regions of strong mitochondrial staining in oncocytic tissues and regions of high relative intensities of CL species in DESI-MS images. Papillary tumors that presented higher relative abundances of CL also showed mitochondria staining by IHC.

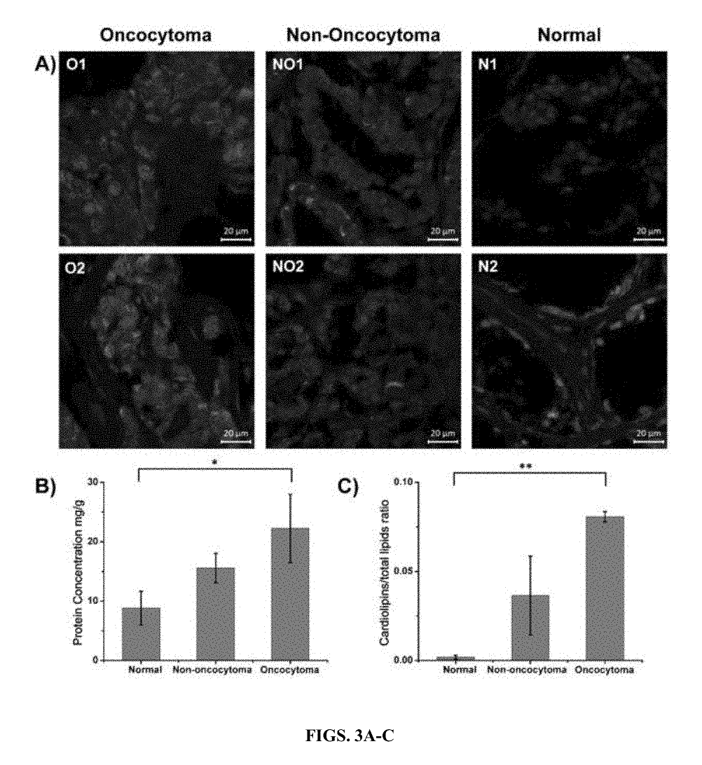

To further investigate the mitochondrial distribution within the cells of thyroid tissues, immunofluorescence staining was performed with an anti-mitochondrial antibody (green) and nuclear staining (blue) in adjacent sections of thyroid tissues (FIG. 3A). Confocal microscopy images obtained for oncocytic tumors show high density staining of mitochondria. A punctate staining pattern showcases accumulation of mitochondria within the cellular cytoplasm in oncocytic tumors. Non-oncocytic follicular carcinoma showed less pronounced accumulation of mitochondria in scattered cells, lower than what was observed for oncocytic tumors, and significantly higher than normal tissue. Using fluorescence intensity measurements, a 50-fold increase in mitochondria/nuclei staining ratio in oncocytic tumors was estimated, and a 6-fold increase in non-oncocytic tumors, when compared to normal thyroid tissues. This could be due to both enlargement and accumulation of mitochondria in oncocytic tumors. Thus, to provide a quantitative assessment of mitochondrial in thyroid tissue, tissues were homogenized and mitochondria was isolated following a specific organelle isolation protocol (FIG. 2A). (23) Quantification of the total protein content in the isolated mitochondria pallet by UV-Vis showed that there is, on average, 2.5 times more protein present in a mitochondrial fraction of oncocytic tumor tissues when compared to normal thyroid tissues. Normal thyroid samples contained in average 8.8 mg of protein/g of tissue sample, while oncocytic tumor samples contained 22.2 mg of protein/g of tissue sample (p value<0.001 using a one-way analysis of variance test) (FIG. 3B). On average, non-oncocytic tumors contained little less than 2 times normal thyroid sample (15.6 mg per sample g).

### Example 3

### Dysregulation of Cardiolipin in Oncocytic Tumors

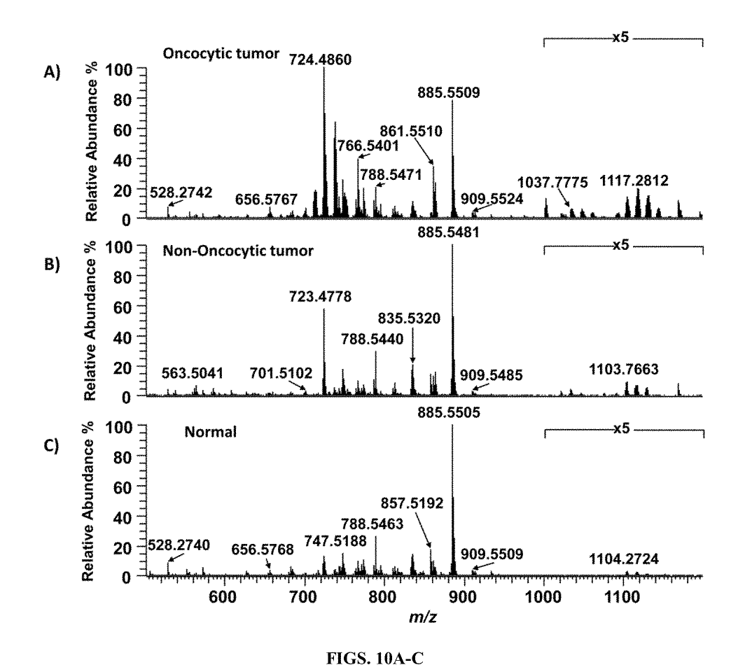

It was hypothesized that the abnormally high relative abundances of CL detected from oncocytic tissues were due to both the accumulation of mitochondria per oncocytic cell, and an alteration in the CL composition of mitochondria membrane. To evaluate this hypothesis, isolated mitochondrial pallets were diluted to the same concentration (3 μg protein/g of tissue) for all tissues and analyzed using the same conditions used for DESI-MS imaging of tissue sections. The mass spectra obtained showed a higher relative intensity of CL species from the mitochondria isolated from oncocytic tumors when compared to non-oncocytic tumors and normal thyroid tissues (FIGS. 10A-C). To compare the CL abundance within the samples, the total ion counts of CL species were normalized to the total lipid counts in the spectra obtained from isolated mitochondria. The average normalized value was 0.081 for oncocytic tumors, 0.037 for non-oncocytic tumors, and 0.002 for normal tissue (FIG. 3C), which allows discrimination between these groups with statistical significance (p value<0.001 using a one-way analysis of variance test). These results confirm that besides mitochondria accumulation, an alteration in the CL composition of the mitochondrial membrane occurs in oncocytic thyroid cells. These biological phenomena collectively contribute to the abnormally high relative intensities and diversity of CL species detected directly from oncocytic tumor tissue in our DESI-MS imaging experiments.

### Example 4

### Lipids as Molecular Markers of Oncocytic Tumors

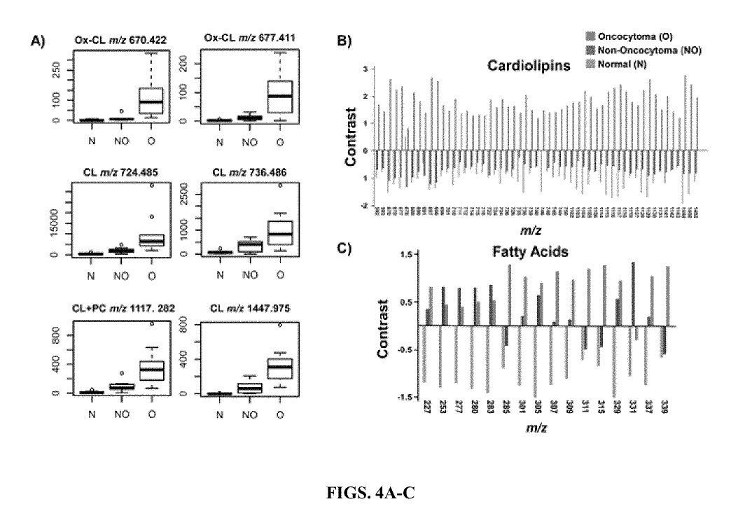

To evaluate if the changes in abundance of CL and other lipid species observed in DESI-MS images and mass spectra obtained were statistically significant, significance analysis of microarrays (SAM) statistical analysis was applied to the complex DESI-MSI dataset. Mass spectral data was extracted from regions of interest of a single predominant histological composition for all samples investigated (e.g. only cancer cells or only follicular cells). SAM identifies if the change in the abundance of a molecular ion (m/z value) is statistically significant between the three different phenotypes by computing a contrast value that measures the average change in the peak intensity for that m/z between the groups. (24) Repeated permutations were used to determine whether the change is significantly related to the phenotype and to estimate the percentage of molecular ions identified by chance, the false discovery rate (FDR). The mean intensity value for all samples for a certain m/z was set to zero, so that the contrast values obtained represent the mean fold increase (positive contrast) or decrease (negative contrast) for the groups when compared to the overall mean intensity value. From all the ions detected (m/z 100-1500) for all the samples analyzed, 219 different molecular ions were selected with FDR<5% (Table 3). As expected, ions corresponding to CL, ox-CL, and CL+PC or CL+DG presented the most significant changes in average abundances between the three groups by SAM analysis, as presented using box plots for selected ions in FIG. 4A. For example, the singly charged CL (20:4/18:2/18:2/16:0) detected at m/z 1447.975 presented the highest contrast values of −1.927 for normal tissue, −0.845 for non-oncocytic tumors, and +2.772 for oncocytic tumors (FDR=0). FIG. 4B showed the overall trend in contrast values obtained for the cardiolipin species selected by SAM (FDR<5%). As observed, all CL species present positive values for oncocytic tumors, which demonstrates that these lipids are significant for discriminating oncocytic tumors from non-oncocytic tumors and normal thyroid tissues. The remaining GP selected by SAM (FDR<5%) including PI, PE and PG presented no clear trends in contrast values within the three groups.

TABLE 3
All the ions detected in m/z 100-1500 range using DESI-MS with
FDR < 5%.

| m/z | | | | | FDR |
|---|---|---|---|---|---|
| value | Score(d) | N | NO | O | (<5%) |
| 191.002 | 1.439 | 2.15 | −1 | −1.15 | 0 |
| 365.348 | 1.342 | −1.772 | 1.719 | 0.053 | 0 |
| 445.317 | 1.295 | −1.166 | 1.922 | −0.756 | 0 |
| 656.572 | 1.316 | −1.455 | −0.419 | 1.874 | 0 |
| 669.916 | 1.745 | −1.532 | −1.091 | 2.623 | 0 |
| 670.419 | 1.492 | −1.239 | −1.005 | 2.244 | 0 |
| 670.602 | 1.135 | −1.337 | −0.24 | 1.577 | 0 |
| 677.405 | 1.566 | −1.381 | −0.974 | 2.356 | 0 |
| 687.543 | 1.211 | 1.487 | 0.186 | −1.672 | 0 |
| 689.432 | 1.407 | −1.151 | −0.98 | 2.131 | 0 |
| 690.447 | 1.185 | −1.023 | −0.779 | 1.802 | 0 |
| 694.387 | 1.249 | 1.915 | −0.744 | −1.172 | 0 |
| 697.428 | 1.77 | −1.432 | −1.251 | 2.683 | 0 |
| 697.916 | 1.663 | −1.376 | −1.164 | 2.54 | 0 |
| 700.513 | 1.214 | −1.334 | 1.744 | −0.41 | 0 |
| 709.563 | 1.156 | −1.741 | 1.012 | 0.729 | 0 |
| 710.471 | 1.288 | −1.25 | −0.646 | 1.896 | 0 |
| 722.463 | 1.236 | −0.965 | −0.901 | 1.866 | 0 |
| 724.485 | 1.281 | −1.222 | −0.66 | 1.883 | 0 |
| 724.532 | 1.307 | −0.473 | 1.901 | −1.429 | 0 |
| 730.516 | 1.213 | −1.535 | −0.564 | 2.099 | 0 |
| 736.486 | 1.424 | −1.536 | −0.505 | 2.041 | 0 |
| 737.514 | 1.473 | 1.396 | 0.793 | −2.188 | 0 |
| 742.523 | 1.657 | −1.783 | −0.595 | 2.377 | 0 |
| 744.55 | 1.417 | −1.693 | −0.256 | 1.949 | 0 |
| 745.505 | 1.144 | −1.492 | 0.018 | 1.474 | 0 |
| 746.563 | 1.315 | −1.236 | −0.747 | 1.983 | 0 |
| 768.541 | 1.397 | −1.629 | −0.318 | 1.947 | 0 |
| 791.547 | 1.145 | −1.321 | −0.289 | 1.61 | 0 |
| 808.509 | 1.476 | 2.055 | −0.248 | −1.807 | 0 |
| 811.551 | 1.31 | 1.619 | 0.152 | −1.772 | 0 |
| 833.518 | 1.22 | 0.039 | 1.559 | −1.599 | 0 |
| 846.51 | 1.308 | 2.069 | −0.838 | −1.231 | 0 |
| 857.506 | 1.65 | −0.065 | 2.165 | −2.101 | 0 |
| 871.536 | 1.3 | −1.215 | 1.958 | −0.743 | 0 |
| 1102.277 | 1.193 | −1.156 | −0.607 | 1.763 | 0 |
| 1103.278 | 1.258 | −1.41 | −0.375 | 1.785 | 0 |
| 1104.273 | 1.515 | −1.594 | −0.599 | 2.194 | 0 |
| 1105.283 | 1.333 | −1.244 | −0.734 | 1.978 | 0 |
| 1114.26 | 1.134 | −1.157 | −0.516 | 1.673 | 0 |
| 1115.268 | 1.5 | −1.611 | −0.555 | 2.166 | 0 |
| 1116.274 | 1.596 | −1.72 | −0.575 | 2.295 | 0 |
| 1117.281 | 1.659 | −1.689 | −0.733 | 2.422 | 0 |
| 1118.299 | 1.471 | −1.398 | −0.778 | 2.175 | 0 |
| 1119.294 | 1.184 | −0.947 | −0.833 | 1.78 | 0 |
| 1128.272 | 1.513 | −1.535 | −0.69 | 2.225 | 0 |
| 1129.28 | 1.774 | −1.709 | −0.917 | 2.625 | 0 |
| 1130.292 | 1.387 | −1.206 | −0.868 | 2.074 | 0 |
| 1141.281 | 1.285 | −1.198 | −0.798 | 1.997 | 0 |
| 1167.727 | 1.442 | −1.494 | −0.602 | 2.096 | 0 |
| 1169.74 | 1.278 | −1.164 | −0.737 | 1.9 | 0 |
| 1348.191 | 1.312 | 1.99 | −1.045 | −0.944 | 0 |
| 1447.969 | 1.896 | −1.927 | −0.845 | 2.772 | 0 |
| 1449.98 | 1.64 | −1.582 | −0.837 | 2.419 | 0 |
| 1451.994 | 1.305 | −1.122 | −0.833 | 1.955 | 0 |
| 176.024 | 1.082 | −1.367 | −0.067 | 1.434 | 0.672 |
| 256.237 | 1.088 | −1.621 | 0.93 | 0.69 | 0.672 |
| 305.233 | 1.054 | −1.565 | 0.649 | 0.916 | 0.672 |
| 308.268 | 1.073 | −1.329 | −0.12 | 1.449 | 0.672 |
| 369.254 | 1.128 | −1.576 | 0.218 | 1.358 | 0.672 |
| 592.364 | 1.13 | −0.986 | −0.695 | 1.681 | 0.672 |
| 655.499 | 1.052 | −1.541 | 0.495 | 1.046 | 0.672 |
| 669.432 | 1.055 | −0.991 | −0.579 | 1.569 | 0.672 |
| 680.413 | 1.067 | −0.995 | −0.786 | 1.781 | 0.672 |
| 683.538 | 1.054 | −1.422 | 0.081 | 1.341 | 0.672 |
| 698.424 | 1.089 | −0.931 | −0.725 | 1.656 | 0.672 |
| 707.55 | 1.048 | −1.562 | 1.014 | 0.547 | 0.672 |
| 723.984 | 1.07 | −0.893 | −0.699 | 1.592 | 0.672 |
| 725.505 | 1.081 | −0.852 | −0.758 | 1.61 | 0.672 |
| 726.492 | 1.103 | −0.956 | −0.681 | 1.637 | 0.672 |
| 747.518 | 1.129 | −1.32 | −0.246 | 1.566 | 0.672 |
| 749.997 | 1.105 | −1.06 | −0.584 | 1.644 | 0.672 |
| 797.543 | 1.094 | −0.977 | 1.632 | −0.655 | 0.672 |
| 810.526 | 1.132 | 1.352 | 0.206 | −1.558 | 0.672 |
| 849.659 | 1.103 | 1.633 | −0.163 | −1.47 | 0.672 |
| 859.533 | 1.083 | −0.347 | 1.542 | −1.195 | 0.672 |
| 899.565 | 1.078 | −1.02 | −0.597 | 1.617 | 0.672 |
| 1090.762 | 1.087 | −0.988 | −0.656 | 1.644 | 0.672 |
| 1091.258 | 1.105 | −0.916 | −0.754 | 1.67 | 0.672 |
| 1127.267 | 1.096 | −0.974 | −0.692 | 1.666 | 0.672 |
| 1350.192 | 1.064 | 1.619 | −0.837 | −0.782 | 0.672 |
| 267.074 | 1.039 | −1.221 | −0.255 | 1.477 | 1.13 |
| 329.246 | 1.036 | −1.531 | 0.573 | 0.958 | 1.13 |
| 330.24 | 1.034 | −1.41 | 1.27 | 0.14 | 1.13 |
| 415.235 | 1.024 | −1.413 | 1.236 | 0.177 | 1.13 |
| 628.546 | 1.026 | −1.104 | −0.372 | 1.476 | 1.13 |
| 671.586 | 1.023 | −1.156 | −0.304 | 1.46 | 1.13 |
| 699.424 | 1.01 | −0.823 | −0.833 | 1.656 | 1.13 |
| 701.525 | 1.031 | 1.495 | −0.393 | −1.101 | 1.13 |
| 748.979 | 1.01 | −0.934 | −0.593 | 1.527 | 1.13 |
| 767.545 | 1.035 | −1.211 | −0.226 | 1.436 | 1.13 |
| 772.532 | 1.018 | −1.101 | −0.37 | 1.471 | 1.13 |
| 847.647 | 1.033 | 1.334 | 0.116 | −1.45 | 1.13 |
| 919.746 | 1.003 | 1.536 | −0.842 | −0.694 | 1.13 |
| 1033.747 | 1.011 | −1.193 | −0.213 | 1.406 | 1.13 |
| 1106.277 | 1.022 | −0.783 | −0.756 | 1.539 | 1.13 |
| 162.963 | 0.963 | −1.103 | 1.392 | −0.289 | 1.501 |
| 268.807 | 0.959 | 1.458 | −0.716 | −0.743 | 1.501 |
| 371.295 | 0.994 | −1.451 | 0.405 | 1.046 | 1.501 |
| 392.27 | 0.962 | −1.183 | 1.307 | −0.124 | 1.501 |
| 593.371 | 0.964 | −0.791 | −0.65 | 1.44 | 1.501 |
| 651.475 | 0.961 | −1.275 | 0.061 | 1.214 | 1.501 |
| 701.491 | 0.984 | −0.81 | −0.659 | 1.469 | 1.501 |
| 712.487 | 0.986 | −0.837 | −0.631 | 1.467 | 1.501 |
| 735.481 | 0.987 | −1.136 | −0.253 | 1.389 | 1.501 |
| 738.989 | 0.995 | −0.84 | −0.64 | 1.48 | 1.501 |
| 740.523 | 0.969 | −0.948 | −0.472 | 1.421 | 1.501 |
| 835.544 | 0.992 | −0.54 | 1.464 | −0.924 | 1.501 |
| 868.479 | 0.981 | 1.545 | −0.48 | −1.066 | 1.501 |
| 873.547 | 0.979 | −0.889 | 1.586 | −0.696 | 1.501 |
| 876.097 | 0.964 | −0.836 | −0.623 | 1.459 | 1.501 |
| 877.594 | 0.962 | −0.828 | −0.631 | 1.458 | 1.501 |
| 890.598 | 0.965 | −1.066 | 1.42 | −0.354 | 1.501 |
| 915.477 | 1.002 | 1.422 | 0.09 | −1.513 | 1.501 |
| 959.484 | 0.957 | −1.053 | 1.449 | −0.396 | 1.501 |
| 1046.753 | 0.965 | −1.069 | −0.324 | 1.393 | 1.501 |
| 1131.303 | 0.999 | −0.768 | −0.734 | 1.502 | 1.501 |
| 283.243 | 0.947 | −1.41 | 0.872 | 0.537 | 1.902 |
| 307.276 | 0.921 | −1.236 | 0.097 | 1.139 | 1.902 |
| 331.269 | 0.948 | −1.055 | 1.346 | −0.29 | 1.902 |
| 443.289 | 0.914 | −1.26 | 1.089 | 0.171 | 1.902 |
| 506.28 | 0.932 | −0.731 | 1.397 | −0.665 | 1.902 |
| 616.403 | 0.93 | −0.827 | −0.58 | 1.407 | 1.902 |
| 653.485 | 0.951 | −1.416 | 0.59 | 0.826 | 1.902 |
| 677.492 | 0.955 | −1.435 | 0.714 | 0.721 | 1.902 |
| 681.515 | 0.952 | −1.377 | 0.336 | 1.041 | 1.902 |
| 711.48 | 0.936 | −0.943 | −0.423 | 1.365 | 1.902 |
| 716.516 | 0.949 | −1.325 | 0.21 | 1.115 | 1.902 |
| 733.559 | 0.944 | −1.416 | 0.424 | 0.993 | 1.902 |
| 748.466 | 0.926 | −0.972 | −0.443 | 1.415 | 1.902 |
| 748.526 | 0.955 | −1.113 | −0.213 | 1.326 | 1.902 |
| 766.56 | 0.94 | −1.264 | 0.105 | 1.159 | 1.902 |
| 773.534 | 0.954 | −1.338 | 0.251 | 1.087 | 1.902 |
| 775.545 | 0.953 | −1.296 | 0.138 | 1.158 | 1.902 |
| 777.545 | 0.945 | −0.909 | −0.499 | 1.408 | 1.902 |
| 865.092 | 0.947 | −0.882 | −0.532 | 1.414 | 1.902 |
| 865.625 | 0.918 | −0.818 | −0.555 | 1.372 | 1.902 |
| 866.599 | 0.946 | −0.711 | −0.722 | 1.433 | 1.902 |
| 878.644 | 0.942 | −0.748 | −0.691 | 1.439 | 1.902 |
| 887.561 | 0.909 | −1.302 | 0.981 | 0.321 | 1.902 |
| 888.556 | 0.944 | −1.364 | 0.989 | 0.375 | 1.902 |
| 889.574 | 0.951 | −1.42 | 0.624 | 0.796 | 1.902 |
| 1048.793 | 0.937 | −1.165 | −0.215 | 1.38 | 1.902 |
| 1090.265 | 0.935 | −0.781 | −0.641 | 1.422 | 1.902 |
| 1091.757 | 0.923 | −0.821 | −0.575 | 1.396 | 1.902 |
| 1142.292 | 0.941 | −0.732 | −0.701 | 1.432 | 1.902 |
| 111.015 | 0.868 | −0.835 | −0.452 | 1.287 | 2.486 |
| 157.105 | 0.875 | −0.956 | 1.264 | −0.308 | 2.486 |
| 159.101 | 0.906 | −0.824 | −0.677 | 1.502 | 2.486 |
| 253.22 | 0.875 | −1.289 | 0.833 | 0.456 | 2.486 |
| 280.238 | 0.89 | −1.316 | 0.809 | 0.508 | 2.486 |
| 301.224 | 0.894 | −1.254 | 0.219 | 1.035 | 2.486 |
| 315.21 | 0.869 | −0.84 | −0.439 | 1.279 | 2.486 |
| 337.32 | 0.891 | −1.242 | 0.193 | 1.049 | 2.486 |
| 340.209 | 0.896 | −0.905 | −0.407 | 1.312 | 2.486 |
| 509.286 | 0.87 | −1.155 | 1.122 | 0.033 | 2.486 |
| 586.232 | 0.888 | 1.264 | −0.206 | −1.057 | 2.486 |
| 629.483 | 0.899 | −1.236 | 0.162 | 1.074 | 2.486 |
| 630.49 | 0.896 | −1.261 | 0.229 | 1.031 | 2.486 |
| 678.407 | 0.895 | 0.513 | −1.337 | 0.824 | 2.486 |
| 684.598 | 0.906 | −1.348 | 0.56 | 0.788 | 2.486 |
| 691.424 | 0.879 | −0.473 | −0.913 | 1.386 | 2.486 |
| 705.529 | 0.892 | −1.236 | 0.106 | 1.13 | 2.486 |
| 710.568 | 0.879 | −1.373 | 0.768 | 0.605 | 2.486 |
| 714.506 | 0.898 | −0.874 | −0.444 | 1.318 | 2.486 |
| 715.514 | 0.874 | −0.803 | −0.493 | 1.295 | 2.486 |
| 736.647 | 0.901 | −1.13 | 1.208 | −0.077 | 2.486 |
| 744.501 | 0.882 | −0.856 | 1.324 | −0.468 | 2.486 |
| 749.512 | 0.877 | −1.04 | −0.168 | 1.208 | 2.486 |
| 796.531 | 0.878 | −1.319 | 0.643 | 0.676 | 2.486 |
| 881.52 | 0.896 | −0.368 | 1.308 | −0.941 | 2.486 |
| 900.567 | 0.904 | −0.774 | −0.628 | 1.401 | 2.486 |
| 901.787 | 0.898 | −1.37 | 0.721 | 0.648 | 2.486 |
| 1049.294 | 0.889 | −1.191 | −0.11 | 1.302 | 2.486 |
| 121.024 | 0.848 | −1.252 | 0.435 | 0.817 | 3.469 |
| 146.047 | 0.86 | −0.926 | −0.309 | 1.235 | 3.469 |
| 285.271 | 0.842 | −0.878 | −0.412 | 1.29 | 3.469 |
| 339.219 | 0.843 | −0.667 | −0.59 | 1.257 | 3.469 |
| 552.264 | 0.86 | −0.918 | 1.242 | −0.324 | 3.469 |
| 627.476 | 0.83 | −1.076 | 0.003 | 1.073 | 3.469 |
| 631.54 | 0.863 | −0.701 | −0.645 | 1.345 | 3.469 |
| 654.56 | 0.844 | −1.223 | 0.325 | 0.899 | 3.469 |
| 679.495 | 0.865 | −1.213 | 0.209 | 1.004 | 3.469 |
| 683.438 | 0.839 | −0.734 | −0.546 | 1.28 | 3.469 |
| 684.442 | 0.835 | −0.659 | −0.613 | 1.272 | 3.469 |
| 710.631 | 0.862 | −1.077 | −0.082 | 1.159 | 3.469 |
| 713.996 | 0.862 | −0.693 | −0.592 | 1.286 | 3.469 |
| 731.539 | 0.838 | −1.249 | 0.297 | 0.951 | 3.469 |
| 746.512 | 0.865 | −1.285 | 0.526 | 0.759 | 3.469 |
| 747.988 | 0.867 | −0.765 | −0.638 | 1.403 | 3.469 |
| 838.562 | 0.831 | 1.113 | −0.077 | −1.037 | 3.469 |
| 847.567 | 0.841 | −1.124 | 1.086 | 0.037 | 3.469 |
| 877.109 | 0.853 | −0.689 | −0.606 | 1.295 | 3.469 |
| 899.771 | 0.85 | −1.276 | 0.779 | 0.497 | 3.469 |
| 905.499 | 0.857 | −0.732 | −0.552 | 1.283 | 3.469 |
| 1047.268 | 0.852 | −0.807 | −0.466 | 1.272 | 3.469 |
| 143.12 | 0.818 | −0.819 | 1.202 | −0.383 | 4.402 |
| 227.189 | 0.808 | −1.185 | 0.357 | 0.828 | 4.402 |
| 277.215 | 0.816 | −1.202 | 0.798 | 0.404 | 4.402 |
| 309.278 | 0.813 | −1.114 | 0.14 | 0.974 | 4.402 |
| 311.292 | 0.805 | −0.712 | −0.485 | 1.197 | 4.402 |
| 535.49 | 0.8 | −1.108 | 0.16 | 0.948 | 4.402 |
| 567.541 | 0.815 | −0.752 | −0.527 | 1.279 | 4.402 |
| 587.503 | 0.821 | −1.159 | 0.221 | 0.938 | 4.402 |
| 594.367 | 0.807 | −0.618 | −0.596 | 1.214 | 4.402 |
| 607.476 | 0.819 | −1.05 | −0.019 | 1.069 | 4.402 |
| 626.542 | 0.827 | −1.233 | 0.512 | 0.721 | 4.402 |
| 658.517 | 0.809 | −0.874 | 1.2 | −0.326 | 4.402 |
| 659.575 | 0.813 | −0.921 | −0.249 | 1.17 | 4.402 |
| 698.496 | 0.822 | −1.242 | 0.666 | 0.576 | 4.402 |
| 705.48 | 0.829 | −0.891 | −0.54 | 1.43 | 4.402 |
| 740.003 | 0.806 | −0.623 | −0.579 | 1.202 | 4.402 |
| 761.527 | 0.812 | −1.09 | 0.059 | 1.031 | 4.402 |
| 795.562 | 0.823 | −0.896 | 1.186 | −0.289 | 4.402 |
| 798.537 | 0.829 | −1.216 | 0.868 | 0.348 | 4.402 |
| 882.517 | 0.818 | −0.439 | 1.229 | −0.79 | 4.402 |
| 1032.742 | 0.807 | −0.942 | −0.193 | 1.135 | 4.402 |
| 1059.76 | 0.818 | −0.797 | −0.45 | 1.247 | 4.402 |
| 1143.292 | 0.801 | −0.655 | −0.558 | 1.213 | 4.402 |

An evident trend of positive values for FA was observed for both non-oncocytic tumors and oncocytic tumors when compared to normal tissues, which consistently showed negative contrast values (FIG. 4C). Interestingly, when comparing the differences of oncocytic and non-oncocytic tumors, higher contrast values were seen for FAs with higher molecular weights (longer carbon chains or less double bonds) in the oncocytic tumor, while higher contrast values were observed for FAs with lower molecular weights in the non-oncocytic tumor. The only two exceptions observed were for FA (14:1) (m/z 227.203) and FA (20:1) (m/z 331.266). These statistically significant results indicate that aberrant FA metabolism also plays an important role in thyroid tumors.

### Example 5

### Materials and Methods

Banked Human Thyroid Tissues. 30 frozen human tissue specimens including Hurthle cell adenomas, carcinomas, non-oncocytic tumors and normal thyroidtissue were obtained from Cooperative Human Tissue Network, Baylor College Tissue Bank, and Asterand Biosciences under approved IRB protocol. Samples were stored in a −80° C. freezer until sectioned. Tissue samples were sectioned at 16 μm thick sections using a CryoStar™ NX50 cryostat (Thermo Scientific, San Jose, Calif.). After sectioning, the glass slides were stored in a −80° C. freezer. Prior to MS imaging, the glass slides were dried in a desiccator for approximately 15 min.

DESI-MS imaging. A 2D Omni Spray (Prosolia Inc., Indianapolis, Ind.) coupled to an LTQ-Orbitrap Elite mass spectrometer (Thermo Scientific, San Jose, Calif.) was used for tissue imaging. DESI-MSI was performed in the negative ion mode from m/z 100-1500, using a hybrid LTQ-Orbitrap mass spectrometer which allows for tandem mass spectrometry experiments, high mass accuracy (<5 ppm mass error) and high mass resolution (240,000 resolving power) measurements. The spatial resolution of the imaging experiments was of 150 μm. The histologically compatible solvent system dimethylformamide:acetonitrile (DMF:ACN) 1:1 (v/v) was used for analysis, at a flow rate of 1 μL/min. The N₂ pressure was set to 180 psi. After DESI-MSI, the same tissue section was subjected to H&E staining, and the adjacent slide was subjected to immunohistochemistry for histopathologic evaluation. For ion identification, the Orbitrap analyzer was used for high mass resolution/accuracy measurements using the same tissue sections analyzed with the ion trap. Tandem MS analyses were performed using both the Orbitrap and the linear ion trap for mass analysis.

Histopathology. The same tissue sections analyzed by DESI-MSI were subjected afterward to standard H&E staining protocol. Pathologic evaluation was performed using light microscopy. Regions of clear diagnosis of cancer and normal thyroid tissue were assigned in the glass slides.

Immunohistochemistry and light microscopy. Hydrate formalin fixed tissue sections were blocked 3% H₂O₂ in water for 10 minutes. After washing, antigen was retrieved with 10 mM Citrate Buffer (pH 6.0) in a microwave oven for 3 minutes at 100% power followed by 10 minutes at 50% power. After cooling for 20 minutes, the slide were then water washed. Non-specific antibody binding was blocked by incubating slides with Casein in buffer for 10 minutes. The slides were then drained and incubated with primary Human Mitochondria monoclonal antibody (clone 113-1) at a 1:2000 dilution overnight at 4° C. The slides were then washed with buffer for five minutes and incubated with biotinylated rabbit-anti-mouse F(ab)′ at a 1:250 dilution for 15 minutes at room temperature. Next, the slides were again washed with buffer for five minutes. Slides were then incubated with SA-HRP (Biocare) for 30 minutes at room temperature. The slides were again washed with buffer for five minutes, then incubated with Sigma Tablet DAB monitoring staining development. All the H&E and IHC stained slides were scanned by using the Aperio ScanScope imaging platform (Aperio Technologies, Vista, Calif., USA) with a 20× objective at a spatial sampling period of 0.47 μm per pixel. Whole slides images (WSI) were viewed and analyzed by using desktop personal computers equipped with the free ScanScope software.

Immunofluorescence and Confocal Microscopy. Formalin fixed tissues were subject to antigen retrieval in 10 mM citrate buffer pH 6.0 heated for 3 min in a microwave at 100% power and cooled for 20 min. Following PBS wash, tissues were blocked with Background Sniper (Biocare Medical, Concord, Calif., USA) for 15 min followed by staining with Alexa Fluor 488 conjugated anti-mitochondrial antibody (MAB1273A4) diluted 1:75 in DaVinci green antibody diluent (Biocare Medical) and incubated for 2 h at room temperature. Stained tissues were washed 3× in PBS wash buffer, DAPI (4′,6-diamidino-2-phenylindole dihydrochloride) counterstained and mounted in ProLong Gold Antifade mounting media (Thermo Fisher). Immunofluorescent images were acquired on a Zeiss LSM880 confocal microscope using the Plan-Apochromat 63 x or 100x objective lens as indicated.

Mitochondria isolation and analysis. Mitochondria isolation was carried out with 10 different thyroid tissue samples: 3 normal tissues, 3 non-oncocytoma and 4 oncocytoma. Tissues were stored at −80° C. before isolation. Small portions of tissue were weighed and then suspended in ice cold H medium (0.3 M sucrose, 1 mM EDTA, 5 mM MOPS, 5 mM KH₂PO4, pH 7.4 at 4° C.) containing 0.1% BSA. All of the remaining steps were performed at 4° C. or on ice. Tissues were homogenized using Teflon Thomas homogenizer (Thomas scientific, Swedesboro, N.J., USA) with 4 up-down strokes of the pestle attached to a drill press operating at 860 rpm. Samples were then centrifuged at 1500 g for 10 min. Suspensions were transferred to new tubes. Collected suspensions were combined and centrifuged at 10,000 g for 10 min to pellet mitochondrial fraction. Mitochondrial pellet was recovered and the leftover samples were centrifuged again for obtaining a higher yield. All the collected mitochondrial fraction pellets were combined and re-suspended in H-medium. The isolated mitochondria were kept frozen in −80° C. until analysis.

Quantification was done using BCA Protein Assays kit (Thermo Scientific, Austin, Tex., USA) according to the manufacturer's protocol. Absorbance measurements were carried out with SpectraMax M3 Molecular Devices (Molecular Devices, Silicon Valley, Calif., USA) at 562 nm wavelengths. Concentrations were measured by comparing absorbance to standard protein curving using different concentration of BSA. Total lipid extraction was carried out by Bligh-Dyer method. (32)

Statistical Analysis. Regions of interest in the 2D raw data obtained by DESI-MSI were selected, converted to text files, and imported to R language for statistical analysis. To reduce complexity and account for small differences in registration between spectra, the data were binned to m/z 0.01. The SAM method was applied using the glmnet package in the CRAN R language library.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. Tallini G (1998) Oncocytic tumours. Virchows Archiv-an International Journal of Pathology 433(1):5-12.
2. Baris O, et al. (2004) Transcriptional profiling reveals coordinated up-regulation of oxidative metabolism genes in thyroid oncocytic tumors. Journal of Clinical Endocrinology & Metabolism 89(2): 994-1005.
3. Gasparre G, et al. (2007) Disruptive mitochondrial DNA mutations in complex I subunits are markers of oncocytic phenotype in thyroid tumors. Proceedings of the National Academy of Sciences of the United States of America 104(21):9001-9006.
4. Mejia E M, Nguyen H, & Hatch G M (2014) Mammalian cardiolipin biosynthesis. Chemistry and Physics of Lipids 179:11-16.
5. Ji J, et al. (2012) Lipidomics identifies cardiolipin oxidation as a mitochondrial target for redox therapy of brain injury. Nature Neuroscience 15(10):1407-1413.
6. Sapandowski A, et al. (2015) Cardiolipin composition correlates with prostate cancer cell proliferation. Molecular and Cellular Biochemistry 410(1-2): 175-185.
7. Norris J L & Caprioli R M (2013) Analysis of Tissue Specimens by Matrix-Assisted Laser Desorption/Ionization Imaging Mass Spectrometry in Biological and Clinical Research. Chemical Reviews 113(4):2309-2342.
8. Wu C, Dill A L, Eberlin L S, Cooks R G, & Ifa D R (2013) Mass spectrometry imaging under ambient conditions. Mass Spectrometry Reviews 32(3):218-243.
9. Wiseman J M, Ifa D R, Song Q Y, & Cooks R G (2006) Tissue imaging at atmospheric pressure using desorption electrospray ionization (DESI) mass spectrometry. Angew. Chem.-Int. Ed. 45(43):7188-7192.
10. Takats Z, Wiseman J M, Gologan B, & Cooks R G (2004) Mass spectrometry sampling under ambient conditions with desorption electrospray ionization. Science 306(5695):471-473.
11. Jarmusch A K, et al. (2016) Lipid and metabolite profiles of human brain tumors by desorption electrospray ionization-MS. Proceedings of the National Academy of Sciences of the United States of America 113(6): 1486-1491.
12. Eberlin L S, et al. (2012) Classifying Human Brain Tumors by Lipid Imaging with Mass Spectrometry. Cancer Research 72(3):645-654.
13. Eberlin L S, et al. (2014) Molecular assessment of surgical-resection margins of gastric cancer by mass-spectrometric imaging. Proceedings of the National Academy of Sciences of the United States of America 111(7):2436-2441.
14. Calligaris D, et al. (2014) Application of desorption electrospray ionization mass spectrometry imaging in breast cancer margin analysis. Proceedings of the National Academy of Sciences of the United States of America 111(42): 15184-15189.
15. Guenther S, et al. (2015) Spatially Resolved Metabolic Phenotyping of Breast Cancer by Desorption Electrospray Ionization Mass Spectrometry. Cancer Research 75(9):1828-1837.
16. Ifa D R & Eberlin L S (2016) Ambient Ionization Mass Spectrometry for Cancer Diagnosis and Surgical Margin Evaluation. Clinical Chemistry 62(1):111-123.
17. Eberlin L S, Ferreira C R, Dill A L, Ifa D R, & Cooks R G (2011) Desorption electrospray ionization mass spectrometry for lipid characterization and biological tissue imaging. Biochimica Et Biophysica Acta-Molecular and Cell Biology of Lipids 1811(11):946-960.
18. Eberlin L S, et al. (2014) Alteration of the lipid profile in lymphomas induced by MYC overexpression. Proceedings of the National Academy of Sciences of the United States of America 111(29): 10450-10455.
19. Hsu F F, et al. (2005) Structural characterization of cardiolipin by tandem quadrupole and multiple-stage quadrupole ion-trap mass spectrometry with electrospray ionization. Journal of the American Society for Mass Spectrometry 16(4): 491-504.
20. Han X L, Yang K, Yang J Y, Cheng H, & Gross R W (2006) Shotgun lipidomics of cardiolipin molecular species in lipid extracts of biological samples. Journal of Lipid Research 47(4):864-879.
21. Kim J, Minkler P E, Salomon R G, Anderson V E, & Hoppel C L (2011) Cardiolipin: characterization of distinct oxidized molecular species. Journal of Lipid Research 52(1):125-135.
22. Pasilis S P, Kertesz V, & Van Berkel G J (2008) Unexpected analyte oxidation during desorption electrospray ionization-mass spectrometry. Analytical Chemistry 80(4):1208-1214.
23. Frezza C, Cipolat S, & Scorrano L (2007) Organelle isolation: functional mitochondria from mouse liver, muscle and cultured filroblasts. Nature Protocols 2(2):287-295.
24. Storey J D & Tibshirani R (2003) Statistical significance for genomewide studies. Proceedings of the National Academy of Sciences of the United States of America 100(16):9440-9445.
25. Gohil V M & Greenberg M L (2009) Mitochondrial membrane biogenesis: phospholipids and proteins go hand in hand. Journal of Cell Biology 184(4):468-472.
26. Wallace D C (2005) A mitochondrial paradigm of metabolic and degenerative diseases, aging, and cancer: A dawn for evolutionary medicine. Annual Review of Genetics, Annual Review of Genetics), Vol 39, pp 359-407.
27. Toyokuni S, Okamoto K, Yodoi J, & Hiai H (1995) PERSISTENT OXIDATIVE STRESS IN CANCER. Febs Letters 358(1):1-3.
28. Kagan V E, et al. (2005) Cytochrome c acts as a cardiolipin oxygenase required for release of proapoptotic factors. Nature Chemical Biology 1(4):223-232.
29. Gasparre G, Porcelli A M, Lenaz G, & Romeo G (2013) Relevance of Mitochondrial Genetics and Metabolism in Cancer Development. Cold Spring Harbor Perspectives in Biology 5(2).
30. Tsybrovskyy O & Roessmann-Tsybrovskyy M (2009) Oncocytic versus mitochondrion-rich follicular thyroid tumours: should we make a difference? Histopathology 55(6):665-682.
31. Hoch F L (1988) LIPIDS AND THYROID-HORMONES. Progress in Lipid Research 27(3): 199-270.
32. Gross R W & Han X (2011) Lipidomics at the interface of Structure and Function in Systems Biology. Chemistry & Biology 18(3):284-291.
33. Camarda R, et al. (2016) Inhibition of fatty acid oxidation as a therapy for MYC-overexpressing triple-negative breast cancer. Nature Medicine 22(4):427-+.

1. A method of detecting cancer cells in a thyroid tissue sample comprising:
(a) performing an ambient ionization MS on the sample to obtain a profile for the sample; and
(b) detecting the presence of cancer cells based on the profile.

2. The method of claim 1, further defined an ex vivo method.

3. The method of claim 1, further defined as a method for detecting a thyroid cancer in the subject.

4. The method of claim 3, wherein the thyroid cancer is oncocytic thyroid cancer.

5. The method of claim 3, wherein the thyroid cancer is papillary thyroid cancer, follicular thyroid cancer, anaplastic thyroid cancer, or medullary thyroid cancer.

6. The method of claim 1, wherein the ambient ionization MS comprises DESI-MSI.

7. The method of claim 1, wherein performing ambient ionization comprises measuring a level of a lipid and/or metabolite in the sample.

8. The method of claim 7, wherein the lipid is cardiolipin.

9. The method of claim 6, comprising performing 2D DESI-MSI.

10. The method of claim 9, wherein 2D DESI-MSI comprises a spatial resolution of 500 um to 50 um.

11. The method of claim 1, further comprising obtaining a reference profile and detecting the presence of cancer cells by comparing the profile from the sample to a reference profile.

12-13. (canceled)

14. The method of claim 8, wherein the level of a cardiolipin comprises a level of ox-CL, CL+DG or CL+PC.

15. The method of claim 8, wherein the level of a cardiolipin is a level of one or more of the cardiolipins provided in Table 1 or 2.

16. The method of claim 8, further comprising measuring a level of a plurality of different cardiolipins in the sample.

17-27. (canceled)

28. The method of claim 1, further comprising:
(c) administering at least a first anticancer therapy to a subject identified to have a thyroid cancer.

29-30. (canceled)

31. A method of treating a subject comprising:
(a) selecting a patient determined to have a thyroid cancer in accordance with claim 1; and
(b) administering at least a first anticancer therapy to the subject.

32-33. (canceled)

34. A method of detecting cells exhibiting mitochondrial dysregulation in a subject comprising:
(a) measuring a level of a cardiolipin in a test sample from a subject;
(b) detecting the presence of cells exhibiting mitochondrial dysregulation based on the measured cardiolipin levels.

35-71. (canceled)

72. A method of treating a subject comprising:
(a) selecting a patient determined to have cells exhibiting mitochondrial dysregulation in accordance with claim 34; and
(b) administering at least a first therapy to the subject.

73-75. (canceled)

76. A method comprising measuring levels of a plurality of cardiolipins in a test sample from a subject using 2D DESI-MSI.

77. The method of claim 76, wherein 2D DESI-MSI comprises a resolution of 500 um to 50 um.

78-104. (canceled)

105. A tangible computer-readable medium comprising:
(a) a computer-readable code comprising a database of values corresponding the levels of a plurality of cardiolipins levels in a biological sample; and
(b) a computer-readable code that, when executed, selectively obtains the marker values from the database values and performs a calculation with the selectively obtained marker values.

106-108. (canceled)
